(19) United States
(12) Patent Application Publication
(10) Pub. No.: US 2022/0226502 A1
(43) Pub. Date: Jul. 21, 2022

(54) ADENO-ASSOCIATED VIRUS VECTOR DELIVERY OF CYSTATHIONINE BETA-SYNTHASE (CBS) ENZYME FOR TREATING CBS DEFICIENCY

(71) Applicants: Institute For Cancer Research d/b/aThe Research Institute of Fox Chase Cancer Center, Philadelphia, PA (US); Cornell University, Ithaca, NY (US)

(72) Inventors: Warren Kruger, Philadelphia, PA (US); Hyung-Ok Lee, Philadelphia, PA (US); Stephen Kaminsky, Ithaca, NY (US); Ronald Crystal, Ithaca, NY (US); Dolan Sondhi, Ithaca, NY (US)

(21) Appl. No.: 17/615,382

(22) PCT Filed: Jun. 2, 2020
(86) PCT No.: PCT/US20/35667

§ 371 Date: Nov. 30, 2021

Related U.S. Application Data
(60) Provisional application No. 62/856,168, filed on Jun. 3, 2019.

(51) Int. Cl. A61K 48/00; C12N 15/86; C12N 9/88

(52) U.S. Cl. CPC A61K 48/005; C12N 15/86; C12N 9/88; C12Y 402/01022; C12N 2750/14151; A01K 2267/0306; C07K 2319/42; C12N 2750/14143; A01K 2227/105

(57) ABSTRACT

The present disclosure provides enzyme replacement therapy using gene therapy vectors, such as adeno-associated virus (AAV) vectors expressing human Cystathionine Beta-Synthase (CBS) to reduce the amount of serum homocysteine (Hcy) and increase the amount of downstream metabolites, such as cystathionine and cysteine (Cys), which can be used for treatment of diseases, such as homocystinuria and homocysteine remethylation disorders.

### FIELD

The present disclosure is directed, in part, to enzyme replacement therapy using gene therapy vectors, such as adeno-associated virus (AAV) vectors, expressing Cystathionine Beta-Synthase (CBS) to reduce the amount of serum homocysteine (Hcy) and increase the amount of downstream metabolites, such as cystathionine and cysteine (Cys), which can be used for treatment of diseases such as homocystinuria and homocysteine remethylation disorders.

### BACKGROUND

CBS, a central enzyme in the transsulfuration pathway, plays an essential role in Hcy metabolism in eukaryotes (Mudd et al., The Metabolic and Molecular Basis of Inherited Disease, 2001, 8 Ed., p. 2007-2056, McGraw-Hill, New York). CBS catalyzes Hcy condensation with L-serine to form cystathionine. When CBS activity is dramatically reduced or absent, as a result of certain genetic mutations, Hcy builds up in tissues and blood. CBS enzyme catalyzes a pyridoxal-5′-phosphate (PLP; Vitamin B₆)-dependent condensation of serine and homocysteine to form cystathionine, which is then used to produce cysteine by another PLP-dependent enzyme, cystathionine γ-lyase. In mammalian cells that possess the transsulfuration pathway, CBS occupies a key regulatory position between the re-methylation of Hcy to methionine or its alternative use in the biosynthesis of Cys.

In healthy normal individuals, CBS-mediated conversion of Hcy to cystathionine is the rate-limiting intermediate step of methionine (Met) metabolism to Cys. Vitamin B₆ is an essential coenzyme for this process. In patients with certain genetic mutations in the CBS enzyme, the conversion of Hcy to cystathionine is slowed or absent, resulting in elevations in the serum concentrations of the enzymatic substrate (Hcy) and a corresponding decrease in the serum concentrations of the enzymatic product (cystathionine). The clinical condition of an elevated serum level of Hcy, and its concomitant excretion into the urine, is collectively known as homocystinuria.

The estimates on the prevalence of homocystinuria vary widely. Data from newborn screening and clinical ascertainment provide a range of 1:200,000 to 1:335,000 live births (Mudd et al., The Metabolic and Molecular Basis of Inherited Disease, 2001, 8 Ed., p. 2007-2056, McGraw-Hill, New York). Recent evidence from DNA screening studies of newborns in Denmark, Germany, Norway and the Czech Republic found that the true incidence may be as high as about 1:6,000 (Gaustadnes et al., N. Engl. J. Med., 1999, 1340, 1513; Linnebank et al., Thromb. Haemost., 2001, 85, 986; Refsum et al., Clin. Chem., 2004, 50, 3; and Sokolova et al., Hum. Mutat., 2001, 18, 548). Additionally, recent work has indicated that CBS-deficient homocystinuria (CBSDH) patients exist with either psychiatric or cardiovascular complications, but are currently undiagnosed due to a lack of the characteristic connective tissue defects that are typically instrumental in diagnosis (Li and Stewart, Pathol., 1999, 31, 221; Linnebank et al., J. Inherited Metabol. Dis., 2003, 26, 509; and Maclean et al., Hum. Mutat., 2002, 19, 641). Primary health problems associated with CBSDH include cardiovascular disease with a predisposition to thrombosis, resulting in a high rate of mortality in untreated and partially treated patients; connective tissue problems affecting the ocular system with progressive myopia and lens dislocation (ectopia lentis); connective tissue problems affecting the skeleton characterized by marfanoid habitus, osteoporosis, and scoliosis; and central nervous system problems, including mental retardation and seizures.

Currently, three potential treatment options exist for the treatment of CB SDH: 1) increase of residual activity of CBS activity using pharmacologic doses of Vitamin B₆ in Vitamin B₆-responsive patients; 2) lowering of serum Hcy by a diet with a strict restriction of the intake of Met; and 3) detoxification by betaine-mediated conversion of Hcy into Met, thus lowering serum Hcy concentration. Each of these three therapies is aimed at lowering serum Hcy concentration. The standard treatment for individuals affected with Vitamin B₆ non-responsive CBSDH consists of a Met-restricted diet supplemented with a metabolic formula and Cys (which has become a conditionally essential amino acid in this condition). Intake of meat, dairy products and other food high in natural protein is prohibited. Daily consumption of a poorly palatable, synthetic metabolic formula containing amino acids and micronutrients is required to prevent secondary malnutrition. Supplementation with CYSTADANE® (betaine) is also a standard therapy. Betaine serves as a methyl donor for the remethylation of Hcy to Met catalyzed by betaine-homocysteinemethyltransferase in the liver (Wilcken et al., N. Engl. J. Med., 1983, 309, 448-53). Dietary compliance generally has been poor, even in those medical centers where optimal care and resources are provided, and this non-compliance has major implications on the development of life-threatening complications of homocystinuria. Accordingly, novel approaches are needed in homocystinuria treatment.

### SUMMARY

The present disclosure provides recombinant AAV nucleic acid molecules comprising a CMV early enhancer/chicken beta actin (CAG) promoter operably linked to an exogenous nucleic acid sequence encoding a human Cystathionine β-synthase (hCBS) polypeptide.

The present disclosure also provides methods of preparing the recombinant AAV nucleic acid molecules comprising: amplifying the exogenous nucleic acid sequence encoding the hCBS polypeptide from a source containing the exogenous nucleic acid sequence using a pair of primers; and cloning the amplified exogenous nucleic acid sequence into a pAAV-CAG-containing nucleic acid molecule.

The present disclosure also provides viral vectors comprising the recombinant AAV nucleic acid molecules.

The present disclosure also provides methods of producing a recombinant AAV vector comprising: co-transfecting a host cell with CAG-hCBS DNA surrounded by AAV inverted terminal repeats (ITRs) and a helper nucleic acid molecule that comprises the AAV Rep and Cap sequences and adenovirus helper functions E4, E2a and VA; and culturing the host cell for a period of time sufficient to produce the recombinant AAV vector.

The present disclosure also provides methods of producing a recombinant AAV vector comprising: co-transfecting a host cell with CAG-hCBS DNA surrounded by AAV ITRs and two helper nucleic acid molecules, the first helper nucleic acid molecule comprising the AAV Rep and Cap sequences, and the second helper nucleic acid molecule comprising the adenovirus helper functions E4, E2a and VA; and culturing the host cell for a period of time sufficient to produce the recombinant AAV vector.

The present disclosure also provides methods of producing a recombinant AAV vector comprising: transfecting a host cell with CAG-hCBS DNA surrounded by AAV ITRs, wherein the host cell expresses AAV Cap and Rep proteins and adenoviral replication proteins E2, E4, and VA; and culturing the host cell for a period of time sufficient to produce the recombinant AAV vector.

The present disclosure also provides compositions comprising a recombinant AAV nucleic acid molecule comprising CAG-hCBS DNA surrounded by AAV ITRs and a pharmaceutically acceptable carrier.

The present disclosure also provides compositions comprising an AAV vector encapsidating a recombinant AAV nucleic acid molecule comprising CAG-hCBS DNA surrounded by AAV ITRs and a pharmaceutically acceptable carrier.

The present disclosure also provides methods of preventing or treating a disease, disorder, or condition associated with elevated homocysteine in a subject in need thereof, comprising administering to the subject a composition comprising a recombinant AAV nucleic acid molecule comprising CAG-hCBS DNA surrounded by AAV ITRs encapsidated in a viral vector and a pharmaceutically acceptable carrier.

The present disclosure also provides uses of a composition comprising a recombinant AAV nucleic acid molecule or a viral vector and a pharmaceutically acceptable carrier for the preparation of a medicament for the prevention or treatment of a disease, disorder, or condition associated with elevated homocysteine in a human subject.

The present disclosure also provides uses of a composition comprising a recombinant AAV nucleic acid molecule or a viral vector and a pharmaceutically acceptable carrier for the prevention or treatment of a disease, disorder, or condition associated with elevated homocysteine in a human subject.

### BRIEF DESCRIPTION OF THE DRAWINGS

FIG. 1A shows a map of pAAVrh.10-CAG-hCBS; interspersed tandem repeat (ITR) sequences are required for viral genome replication and packaging; all elements located between the ITR sequences are packaged into the virus.

FIG. 1B shows the experimental scheme of a representative study; time of injection is shown by the red up arrow; ticks above the line show days in which blood was collected; downward arrow shows day of tissue collection for mice with indicated route of injection and dosage; a summary of age, sex, and the method of injection of the mice are shown in the table underneath.

FIG. 2A shows the effect of AAVrh.10-CAG-hCBS on serum tHcy and methionine in mice at −7, 7, 14, and 21 days after injection with indicated dose of virus; IP and IV injections were combined; asterisks indicate P<0.05 compared to D-7 value.

FIG. 2B shows the effect of AAVrh.10-CAG-hCBS on liver and kidney CBS activity in mice 21 days after infection with indicated viral dose; error bar shows SEM; IP and IV injections were combined; control −/− bar indicates saline injected Tg-1287T Cbs^{-/-} mice, while +/+ control bar is activity in wild-type mice.

FIG. 2C shows a comparison of IP versus IV injection on tHcy and methionine; each line represents a single mouse; control mice were saline injected.

FIG. 2D shows a summary of individual mice treatment for experiments in FIG. 2C.

FIG. 2E shows data from FIG. 2C (top) in table form; the middle panel shows percent reduction in tHcy comparing D-7 with D7 and D14 for mock and AAVrh.10-CAG-hCBS injected animals; the bottom panel shows statistical significance of these changes.

FIG. 2F (top) shows data from FIG. 2C (bottom) in table form; the middle panel shows percent reduction in tHcy comparing D-7 with D7 and D14 for mock and AAVrh.10-CAG-hCBS injected animals; the bottom panel shows statistical significance of these changes.

FIG. 3A shows the long-term effects of AAVrh.10-CAG-hCBS on serum tHcy and methionine in treated and control Tg-I278T Cbs^{-/-} mice; error bars show SE; asterisk indicates P<0.005.

FIG. 3B shows data from FIG. 3A in table form.

FIG. 3C shows a Western blot showing liver CBS levels at D78 in control and treated mice; endogenous CBS shows leaky background expression from mutant I278T CBS expressing transgene detected by antibody to an HA tag.

FIG. 4A shows liver and kidney CBS enzyme activity at D78 measured in lysates.

FIG. 4B shows a comparison of alopecia in treated vs. control mice.

FIG. 5A shows an experimental scheme timeline; upticks show days in which blood was collected; red arrow indicates time at which AAV1-CMV-hCBS was delivered into Tg-I278T Cbs^{-/-} mice via a single injection; down arrows show numbers of mice from each group euthanized on each date and the method of AAV injection.

FIG. 5B shows Tg-I278T Cbs^{-/-} mice were injected with PBS or variable numbers of virus by IP, IM or IV; serum tHcy was measured at before (D-7) and a week after injection (D7); asterisks indicate P<0.05 compared D-7 to D7.

FIG. 5C shows mice injected with PBS, 4×10¹², or 8×10¹² virus by IP and CBS activity was measured in the liver and kidney lysates collected at D17; asterisk indicates P<0.05 compared to buffer-injected mice; error bars show SE.

FIG. 6 shows a summary of individual mice treatment with AAV1-CMV-hCBS.

FIG. 7A shows a representative summary and results of a second experiment in which mice on either a CH3 or C57BL6 strain background were either injected or not injected with AAVrh.10.CAG-hCBS; the transgenic mice express the indicated allele CBS mutations.

FIG. 7B shows CBS levels at D27 in control and treated mice; top panel shows total CBS expression; mouse CBS runs slightly slower than hCBS; middle panel shows reactivity using anti-HA antibody; this only expresses transgene expressed CBS; in some control animals, low levels of transgene CBS was detected even though the mice were not on zinc.

FIG. 8 shows a representative summary of individual treatment of Tg-I278T Cbs^{-/-} mice in FIGS. 9A, 9B, and 9C.

FIG. 9A shows the effect of AAVrh.10-CAG-hCBS on weight in Tg-I278T Cbs^{-/-} mice.

FIG. 9B shows the effect of AAVrh.10-CAG-hCBS on serum tHcy in Tg-I278T Cbs^{-/-} mice.

FIG. 9C shows the effect of AAVrh.10-CAG-hCBS on serum methionine in Tg-I278T Cbs^{-/-} mice.

FIG. 10 shows a comparison of alopecia and whiskers in treated vs. control mice.

### DESCRIPTION OF EMBODIMENTS

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “AAV” is an abbreviation for adeno-associated virus, which is a single-stranded DNA parvovirus or self-complementary double-stranded DNA that grows only in cells in which certain functions are provided by a co-infecting helper virus or plasmid.

As used herein, the phrase “AAV nucleic acid molecule” refers to a nucleic acid molecule comprising one or more polynucleotides of interest (transgenes or exogenous nucleic acid sequences, such as those encoding hCBS) that are flanked by AAV ITRs. Such AAV nucleic acid molecules can be replicated and packaged into infectious viral particles when present in a host cell that has been transfected with a helper plasmid encoding and expressing AAV Rep and Cap gene products, as well as other helper gene products.

As used herein, the phrase “AAV vector” refers to a viral particle composed of an AAV capsid protein and an encapsidated polynucleotide comprising at least one AAV nucleic acid molecule (comprising, for example, a polynucleotide sequence encoding hCBS).

As used herein, the term “comprising” may be replaced with “consisting” or “consisting essentially of” in particular embodiments as desired.

As used herein, the term “homolog” refers to a protein or peptide which differs from a naturally occurring protein or peptide (e.g., the “wild-type” protein) by modifications to the naturally occurring protein or peptide, but which maintains the basic protein and side chain structure of the naturally occurring form. Such changes include, but are not limited to: changes in one, few, or even several amino acid side chains; changes in one, few or several amino acids; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A homolog can have enhanced, decreased, changed, or essentially similar properties as compared to the naturally occurring protein or peptide. In some embodiments, truncated CBS proteins, for example having C-terminal deletions of the naturally occurring CBS protein, are included.

As used herein, a “nucleic acid,” a “nucleic acid molecule,” a “nucleic acid sequence,” a “polynucleotide,” or an “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the phrase “operably linked” refers to both expression control sequences that are contiguous with the gene of interest and expression control sequences that act in trans or at a distance to control the gene of interest.

As used herein, the term “serotype” refers to a distinction with respect to an AAV having a capsid which is serologically distinct from other AAV serotypes. Serologic distinctiveness can be determined on the basis of the lack of cross-reactivity between antibodies to the AAV as compared to other AAV. Cross-reactivity is typically measured in a neutralizing antibody assay.

As used herein, the terms “subject” and “patient” are used interchangeably. A subject may include any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates. In some embodiments, the subject is a human.

A used herein, the term “tropism” refers to the ability of an AAV vector to infect one or more specified cell types, but can also encompass how the vector functions to transduce the cell in the one or more specified cell types (i.e., preferential entry of the AAV vector into certain cell or tissue type(s) and/or preferential interaction with the cell surface that facilitates entry into certain cell or tissue types, optionally and followed by expression (e.g., transcription and, optionally, translation) of sequences carried by the AAV vector in the cell.

The present disclosure provides recombinant AAV nucleic acid molecules comprising a CAG promoter operably linked to an exogenous nucleic acid sequence encoding an hCBS polypeptide. In some embodiments, the recombinant AAV nucleic acid molecule is present within a plasmid, bacmid, or baculovirus in order to produce a viral vector. In some embodiments, the recombinant AAV nucleic acid molecule is present within a plasmid. In some embodiments, the recombinant AAV nucleic acid molecule is present within a bacmid. In some embodiments, the recombinant AAV nucleic acid molecule is present within a baculovirus.

In some embodiments, the AAV nucleic acid molecules can contain a full-length AAV 5′ ITR and a full-length 3′ ITR. A shortened version of the 5′ ITR, termed AITR, in which the D-sequence and terminal resolution site (trs) are deleted can also be utilized. In addition, self-complementary AAV (scAAV) nucleic acid molecules can be constructed in which a coding region carried by a recombinant AAV nucleic acid sequence has been designed to form an intra-molecular double-stranded DNA template. Upon infection, rather than waiting for cell-mediated synthesis of the second strand, the two complementary halves of scAAV will associate to form one double stranded DNA (dsDNA) unit that is ready for immediate replication and transcription. In some embodiments, a single-stranded AAV nucleic acid molecule can be used.

In some embodiments, the ITRs can be selected from a source which differs from the AAV source of the capsid. For example, AAV2 ITRs can be selected for use with an AAV capsid having a particular efficiency for a selected cellular receptor, target tissue, or viral target. In some embodiments, the ITR or AITR sequences from AAV2 can be used for convenience and to accelerate regulatory approval. However, ITRs from other AAV sources can also be selected. Where the source of the ITRs is from AAV2 and the AAV capsid is from another AAV source, the resulting vector produced therefrom may be termed pseudotyped. However, other sources of AAV ITRs may be utilized.

In some embodiments, recombinant AAV nucleic acid molecules can comprise the exogenous nucleic acid sequences described herein and one or more flanking AAV ITRs. AAV DNA in the AAV genomes may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, and AAV-13. Production of pseudotyped AAV is disclosed in, for example, PCT Publication No. WO 01/83692. Other types of AAV variants, for example AAV with capsid mutations, are also contemplated (see, Marsic et al., Mol. Therapy, 2014, 22, 1900-1909). In some embodiments, the recombinant AAV nucleic acid molecules are derived from the AAVrh.10 serotype.

In some embodiments, recombinant AAV nucleic acid molecules (such as those used to produce infectious encapsidated AAV particles) comprise an AAV genome. In some embodiments, the genomes of both AAV lack AAV rep and cap DNA (i.e., there is no AAV rep or cap DNA between the ITRs of the genomes). Examples of AAV that may be constructed to comprise the exogenous nucleic acid sequences described herein are included in PCT Publication No. WO 13/016352.

In some embodiments, the AAV nucleic acid molecules described herein are designed for expressing its gene product in specific cell types (such as, hepatocytes). In addition to the AAV 5′ ITR and 3′ ITR, the open reading frame(s) may include tissue-specific regulatory elements or constitutive elements.

The AAV nucleic acid molecules described herein typically contain a CAG promoter sequence as part of the expression control sequences, located between the 5′ ITR sequence and the coding sequence of the exogenous nucleic acid sequence. In some embodiments, the CAG promoter is upstream of the exogenous nucleic acid sequence encoding the hCBS polypeptide. The CAG promoter is a hybrid promoter containing a CMV/chicken beta-actin/rabbit beta-globin splice acceptor sequences (Miyazaki et al., Gene, 1989, 79, 269-77). Additional promoters include, but are not limited to, human alpha 1 anti-trypsin (AAT), human thyroxin binding globulin (TBG), cytomegalovirus (CMV), human GLUT1 promoter, hybrid liver specific promoter (HLP promoter), LP-1 promoter, LSP-1 promoter, chicken beta actin, and mouse beta-actin, and any other promoter that supports expression in the liver.

In some embodiments, the AAV nucleic acid molecules can also contain one or more other appropriate transcription initiation, termination, enhancer sequences, efficient RNA processing signals, such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. Examples of suitable polyA sequences include, but are not limited to, SV40, SV50, bovine growth hormone (bGH), human growth hormone, and synthetic polyAs. Examples of suitable enhancers include, but are not limited to, the alpha fetoprotein enhancer, the TTR minimal promoter/enhancer, LSP (TH-binding globulin promoter/alpha1-microglobulin/bikunin enhancer), amongst others. In some embodiments, the AAV nucleic acid molecules can comprise one or more expression enhancers. In some embodiments, the AAV nucleic acid molecules can contain two or more expression enhancers. These enhancers may be the same or may differ from one another. For example, an enhancer may include an Alpha mic/bik enhancer. This enhancer may be present in two copies which are located adjacent to one another. Alternately, the dual copies of the enhancer may be separated by one or more sequences. In some embodiments, the AAV nucleic acid molecules can further contain an intron such as, for example, the Promega intron. In some embodiments, one or more sequences may be selected to stabilize mRNA. An example of such a sequence is a modified WPRE sequence, which may be engineered upstream of the polyA sequence and downstream of the coding sequence (see, Zanta-Boussif et al, Gene Therapy, 2009, 16, 605-619). These additional elements are operably linked to the coding sequences.

The amino acid sequence for full-length hCBS comprises 551 amino acids: MPSETPQ AEVGPTGCPHRSGPHSAKGSLEKGSPEDKEAKEPLWIRPDAPSRCTWQLGRPASESPHH HTAPAKSPKILPDILKKIGDTPMVRINKIGKKFGLKCELLAKCEFFNAGGSVKDRISLRMI EDAERDGTLKPGDTIIEPTSGNTGIGLALAAAVRGYRCIIVMPEKMSSEKVDVLRALGAE IVRTPTNARFDSPESHVGVAWRLKNEIPNSHILDQYRNASNPLAHYDTTADEILQQCDG KLDMLVASVGTGGTITGIARKLKEKCPGCRIIGVDPEGSILAEPEELNQTEQTTYEVEGIG YDFIPTVLDRTVVDKWFKSNDEEAFTFARMLIAQEGLLCGGSAGSTVAVAVKAAQELQ EGQRCVVILPDSVRNYMTKFLSDRWMLQKGFLKEEDLTEKKPWWWHLRVQELGLSAP LTVLPTITCGHTIEILREKGFDQAPVVDEAGVILGMVTLGNMLSSLLAGKVQPSDQVGK VIYKQFKQIRLTDTLGRLSHILEMDHFALVVHEQIQYHSTGKSSQRQMVFGVVTAIDLLN FVAAQERDQK (SEQ ID NO:1).

In some embodiments, the exogenous nucleic acid sequence encodes an hCBS polypeptide comprising an amino acid sequence at least about 70%, at least about 75%, at least about 80%, at least about 85%, at least about 90%, at least about 95%, at least about 96%, at least about 97%, at least about 98%, or at least about 99% identical to the amino acid sequence of SEQ ID NO:1, or to a biologically active truncation thereof (including non-heme-binding, but catalytically active variants). In some embodiments, the exogenous nucleic acid sequence encodes an hCBS polypeptide comprising an amino acid sequence at least about 70% identical to the amino acid sequence of SEQ ID NO:1. In some embodiments, the exogenous nucleic acid sequence encodes an hCBS polypeptide comprising an amino acid sequence at least about 75% identical to the amino acid sequence of SEQ ID NO:1. In some embodiments, the exogenous nucleic acid sequence encodes an hCBS polypeptide comprising an amino acid sequence at least about 80% identical to the amino acid sequence of SEQ ID NO:1. In some embodiments, the exogenous nucleic acid sequence encodes an hCBS polypeptide comprising an amino acid sequence at least about 85% identical to the amino acid sequence of SEQ ID NO:1. In some embodiments, the exogenous nucleic acid sequence encodes an hCBS polypeptide comprising an amino acid sequence at least about 90% identical to the amino acid sequence of SEQ ID NO:1. In some embodiments, the exogenous nucleic acid sequence encodes an hCBS polypeptide comprising an amino acid sequence at least about 95% identical to the amino acid sequence of SEQ ID NO:1. In some embodiments, the exogenous nucleic acid sequence encodes an hCBS polypeptide comprising an amino acid sequence at least about 96% identical to the amino acid sequence of SEQ ID NO:1. In some embodiments, the exogenous nucleic acid sequence encodes an hCBS polypeptide comprising an amino acid sequence at least about 97% identical to the amino acid sequence of SEQ ID NO:1. In some embodiments, the exogenous nucleic acid sequence encodes an hCBS polypeptide comprising an amino acid sequence at least about 98% identical to the amino acid sequence of SEQ ID NO:1. In some embodiments, the exogenous nucleic acid sequence encodes an hCBS polypeptide comprising an amino acid sequence at least about 99% identical to the amino acid sequence of SEQ ID NO:1. In some embodiments, the exogenous nucleic acid sequence encodes an hCBS polypeptide comprising an amino acid sequence identical to the amino acid sequence of SEQ ID NO:1. In some embodiments, the exogenous nucleic acid sequence encodes an hCBS polypeptide consisting of an amino acid sequence identical to the amino acid sequence of SEQ ID NO:1.

In some embodiments, an hCBS variant can include any combination of the N-terminal deletions or modifications and the C-terminal deletions described herein or in U.S. Pat. No. 8,007,787. In some embodiments, additional modifications can be achieved by modification of other amino acid residues to provide a given percent identity to the wild-type hCBS sequence. In some embodiments, the hCBS variant is a truncated recombinant hCBS (r-hCB SAC) homodimeric enzyme wherein the C-terminal regulatory region has been removed. In some embodiments, any of the hCBS variants described herein can have no more than one or two non-hCBS amino acid residues at the N-terminus (i.e., the variant comprises no more than one or two amino acid residues at the N-terminus that is/are not a residue of the naturally occurring hCBS amino acid sequence at that position).

The AAV nucleic acid molecules described herein contain an exogenous nucleic acid sequence that encodes an hCBS polypeptide, or a homolog thereof. The nucleic acid sequence encoding hCBS and the amino acid sequence encoded thereby are available through GenBank Accession No. L19501, and these sequences are also disclosed in U.S. Pat. No. 5,523,225. A nucleotide sequence coding for hCBS is TCCCGGGCCCGCGACACACGCCCTCGGGGTCG GTCCTCGAGGACGCGCAGGGCCCCCCACCCACCAGGACGCACGTTTCAAGCTCATC AGTAAAGGTTCCTTAAATTCCCGAAGGGCAAGAAGTTAACCAAGTAAAACAGCATC GGAACACCAGGATCCCATGACAGATTCTGTTGTCACGTCTCCTTACAGAGTTTGAGC GGTGCTGAACTGTCAGCACCATCTGTCCGGTCCCAGCATGCCTTCTGAGACCCCCCA GGCAGAAGTGGGGCCCACAGGCTGCCCCCACCGCTCAGGGCCACACTCGGCGAAGG GGAGCCTGGAGAAGGGGTCCCCAGAGGATAAGGAAGCCAAGGAGCCCCTGTGGAT CCGGCCCGATGCTCCGAGCAGGTGCACCTGGCAGCTGGGCCGGCCTGCCTCCGAGT CCCCACATCACCACACTGCCCCGGCAAAATCTCCAAAAATCTTGCCAGATATTCTGA AGAAAATCGGGGACACCCCTATGGTCAGAATCAACAAGATTGGGAAGAAGTTCGGC CTGAAGTGTGAGCTCTTGGCCAAGTGTGAGTTCTTCAACGCGGGCGGGAGCGTGAA GGACCGCATCAGCCTGCGGATGATTGAGGATGCTGAGCGCGACGGGACGCTGAAGC CCGGGGACACGATTATCGAGCCGACATCCGGGAACACCGGGATCGGGCTGGCCCTG GCTGCGGCAGTGAGGGGCTATCGCTGCATCATCGTGATGCCAGAGAAGATGAGCTC CGAGAAGGTGGACGTGCTGCGGGCACTGGGGGCTGAGATTGTGAGGACGCCCACCA ATGCCAGGTTCGACTCCCCGGAGTCACACGTGGGGGTGGCCTGGCGGCTGAAGAAC GAAATCCCCAATTCTCACATCCTAGACCAGTACCGCAACGCCAGCAACCCCCTGGCT CACTACGACACCACCGCTGATGAGATCCTGCAGCAGTGTGATGGGAAGCTGGACAT GCTGGTGGCTTCAGTGGGCACGGGCGGCACCATCACGGGCATTGCCAGGAAGCTGA AGGAGAAGTGTCCTGGATGCAGGATCATTGGGGTGGATCCCGAAGGGTCCATCCTC GCAGAGCCGGAGGAGCTGAACCAGACGGAGCAGACAACCTACGAGGTGGAAGGGA TCGGCTACGACTTCATCCCCACGGTGCTGGACAGGACGGTGGTGGACAAGTGGTTC AAGAGCAACGATGAGGAGGCGTTCACCTTTGCCCGCATGCTGATCGCGCAAGAGGG GCTGCTGTGCGGTGGCAGTGCTGGCAGCACGGTGGCGGTGGCCGTGAAGGCCGCGC AGGAGCTGCAGGAGGGCCAGCGCTGCGTGGTCATTCTGCCCGACTCAGTGCGGAAC TACATGACCAAGTTCCTGAGCGACAGGTGGATGCTGCAGAAGGGCTTTCTGAAGGA GGAGGACCTCACGGAGAAGAAGCCCTGGTGGTGGCACCTCCGTGTTCAGGAGCTGG GCCTGTCAGCCCCGCTGACCGTGCTCCCGACCATCACCTGTGGGCACACCATCGAGA TCCTCCGGGAGAAGGGCTTCGACCAGGCGCCCGTGGTGGATGAGGCGGGGGTAATC CTGGGAATGGTGACGCTTGGGAACATGCTCTCGTCCCTGCTTGCCGGGAAGGTGCAG CCGTCAGACCAAGTTGGCAAAGTCATCTACAAGCAGTTCAAACAGATCCGCCTCAC GGACACGCTGGGCAGGCTCTCGCACATCCTGGAGATGGACCACTTCGCCCTGGTGGT GCACGAGCAGATCCAGTACCACAGCACCGGGAAGTCCAGTCAGCGGCAGATGGTGT TCGGGGTGGTCACCGCCATTGACTTGCTGAACTTCGTGGCCGCCCAGGAGCGGGACC AGAAGTGAAGTCCGGAGCGCTGGGCGGTGCGGAGCGGGCCCGCCACCCTTGCCCAC TTCTCCTTCGCTTTCCTGAGCCCTAAACACACGCGTGATTGGTAACTGCCTGGCCTGG CACCGTTATCCCTGCACACGGCACAGAGCATCCGTCTCCCCTCGTTAACACATGGCT TCCTAAATGGCCCTGTTTACGGCCTATGAGATGAAATATGTGATTTTCTCTAATGTA ACTTCCTCTTAGGATGTTTCACCAAGGAAATATTGAGAGAGAAGTCGGCCAGGTAG GATGAACACAGGCAATGACTGCGCAGAGTGGATTAAAGGCAAAAGAGAGAAGAGT CCAGGAAGGGGCGGGGAGAAGCCTGGGTGGCTCAGCATCCTCCACGGGCTGCGCCG TCTGCTCGGGGCTGAGCTGGCGGGAGCAGTTTGCGTGTTTGGGTTTTTTAATTGAGA TGAAATTCAAATAACCTAAAAATCAATCACTTGAAAGTGAACAATCAGCGGCATTT AGTACATCCAGAAAGTTGTGTAGGCACCACCTCTGTCACGTTCTGGAACATTCTGTC ATCACCCCGTGAAGCAATCATTTCCCCTCCCGTCTTCCTCCTCCCCTGGCAACTGCTG ATCGACTTTGTGTCTCTGTTGTCTAAAATAGGTTTTCCCTGTTCTGGACATTTCATAT AAATGGAATCACACAA (SEQ ID NO:2).

In some embodiments, the exogenous nucleic acid sequence comprises a nucleotide sequence at least about 70%, at least about 75%, at least about 80%, at least about 85%, at least about 90%, at least about 95%, at least about 96%, at least about 97%, at least about 98%, or at least about 99% identical to the nucleotide sequence of SEQ ID NO:2. In some embodiments, the exogenous nucleic acid sequence comprises a nucleotide sequence at least about 70% identical to the nucleotide sequence of SEQ ID NO:2. In some embodiments, the exogenous nucleic acid sequence comprises a nucleotide sequence at least about 75% identical to the nucleotide sequence of SEQ ID NO:2. In some embodiments, the exogenous nucleic acid sequence comprises a nucleotide sequence at least about 80% identical to the nucleotide sequence of SEQ ID NO:2. In some embodiments, the exogenous nucleic acid sequence comprises a nucleotide sequence at least about 85% identical to the nucleotide sequence of SEQ ID NO:2. In some embodiments, the exogenous nucleic acid sequence comprises a nucleotide sequence at least about 90% identical to the nucleotide sequence of SEQ ID NO:2. In some embodiments, the exogenous nucleic acid sequence comprises a nucleotide sequence at least about 95% identical to the nucleotide sequence of SEQ ID NO:2. In some embodiments, the exogenous nucleic acid sequence comprises a nucleotide sequence at least about 96% identical to the nucleotide sequence of SEQ ID NO:2. In some embodiments, the exogenous nucleic acid sequence comprises a nucleotide sequence at least about 97% identical to the nucleotide sequence of SEQ ID NO:2. In some embodiments, the exogenous nucleic acid sequence comprises a nucleotide sequence at least about 98% identical to the nucleotide sequence of SEQ ID NO:2. In some embodiments, the exogenous nucleic acid sequence comprises a nucleotide sequence at least about 99% identical to the nucleotide sequence of SEQ ID NO:2. In some embodiments, the exogenous nucleic acid sequence comprises a nucleotide sequence identical to the nucleotide sequence of SEQ ID NO:2. In some embodiments, the exogenous nucleic acid sequence consists of a nucleotide sequence identical to the nucleotide sequence of SEQ ID NO:2.

Percent sequence identity can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Any amino acid or nucleotide number calculated as a % identity can be rounded up or down, as the case may be, to the closest whole number. Alternately, optimal alignment of sequences for comparison may be conducted by computerized implementations of algorithms (GAP, BESTFIT, BLAST, FASTA, and TFASTA in the Wisconsin Genetics Software Package, Genetics Computer Group (GCG), 575 Science Dr., Madison, Wis.), or by inspection. Additional examples of algorithms for determining percent sequence identity and sequence similarity are the BLAST and BLAST 2.0 algorithms, which are described in Altschul et al., Nuc. Acids Res., 1977, 25, 3389-3402 and Altschul et al., J. Mol. Biol., 1990, 215, 403-410, respectively. Software for performing BLAST analyses is publicly available through the National Center for Biotechnology Information.

The present disclosure also provides methods of preparing the recombinant AAV nucleic acid molecules described herein. In some embodiments, the methods comprise: amplifying the exogenous nucleic acid sequence encoding the hCBS polypeptide from a source containing the exogenous nucleic acid sequence using a pair of primers; and cloning the amplified exogenous nucleic acid sequence into a pAAV-CAG-containing nucleic acid molecule.

In some embodiments, the source containing the exogenous nucleic acid sequence is pUC:ΔHCBS, which is commercially available or can be obtained through RT-PCR of RNA derived from human tissue, or can be chemically synthesized.

In some embodiments, the pair of primers comprises a first primer comprising the nucleotide sequence 5′-CAGTCTCGAACTTAACATGCCTTCTGAGACCCCC-3′ (SEQ ID NO:3) and a second primer comprising the nucleotide sequence 5′-GGGCCCATTACCGAT ACTTCACTTCTGGTCCGCTCC-3′(SEQ ID NO:4).

In some embodiments, the pAAV-CAG-containing nucleic acid molecule is pAAV-CAG-MCS. The pAAV-CAG-MCS plasmid provides the AAV inverted terminal repeats, as well as the expression regulation sequences (CAG promoter and poly A tail). The MCS is a multiple cloning site that facilitates the insertion of the desired gene to be expressed, in this case hCBS. The pAAV-CAG-MCS plasmid is kanamycin resistant. Additional AAV plasmids can be used (see, world wide web at “addgene.org/viral-service/penn-vector-core/”). A representative nucleotide sequence of the pAAV-CAG-MCS construct is: CCTGCAGGCAGCTGCGCGCTCG CTCGCTCACTGAGGCCGCCCGGGCAAAGCCCGGGCGTCGGGCGACCTTTGGTCGCC CGGCCTCAGTGAGCGAGCGAGCGCGCAGAGAGGGAGTGGCCAACTCCATCACTAGG GGTTCCTGCGGCCGCTCTAGAACTAGTCGACATTGATTATTGACTAGTTATTAATAG TAATCAATTACGGGGTCATTAGTTCATAGCCCATATATGGAGTTCCGCGTTACATAA CTTACGGTAAATGGCCCGCCTGGCTGACCGCCCAACGACCCCCGCCCATTGACGTCA ATAATGACGTATGTTCCCATAGTAACGCCAATAGGGACTTTCCATTGACGTCAATGG GTGGAGTATTTACGGTAAACTGCCCACTTGGCAGTACATCAAGTGTATCATATGCCA AGTACGCCCCCTATTGACGTCAATGACGGTAAATGGCCCGCCTGGCATTATGCCCAG TACATGACCTTATGGGACTTTCCTACTTGGCAGTACATCTACGTATTAGTCATCGCTA TTACCATGGTCGAGGTGAGCCCCACGTTCTGCTTCACTCTCCCCATCTCCCCCCCCTC CCCACCCCCAATTTTGTATTTATTTATTTTTTAATTATTTTGTGCAGCGATGGGGGCG GGGGGGGGGGGGGGGCGCGCGCCAGGCGGGGCGGGGCGGGGCGAGGGGCGGGGC GGGGCGAGGCGGAGAGGTGCGGCGGCAGCCAATCAGAGCGGCGCGCTCCGAAAGT TTCCTTTTATGGCGAGGCGGCGGCGGCGGCGGCCCTATAAAAAGCGAAGCGCGCGG CGGGCGGGAGTCGCTGCGCGCTGCCTTCGCCCCGTGCCCCGCTCCGCCGCCGCCTCG CGCCGCCCGCCCCGGCTCTGACTGACCGCGTTACTCCCACAGGTGAGCGGGCGGGA CGGCCCTTCTCCTCCGGGCTGTAATTAGCGCTTGGTTTAATGACGGCTTGTTTCTTTT CTGTGGCTGCGTGAAAGCCTTGAGGGGCTCCGGGAGGGCCCTTTGTGCGGGGGGAG CGGCTCGGGGGGTGCGTGCGTGTGTGTGTGCGTGGGGAGCGCCGCGTGCGGCTCCG CGCTGCCCGGCGGCTGTGAGCGCTGCGGGCGCGGCGCGGGGCTTTGTGCGCTCCGC AGTGTGCGCGAGGGGAGCGCGGCCGGGGGCGGTGCCCCGCGGTGCGGGGGGGGCT GCGAGGGGAACAAAGGCTGCGTGCGGGGTGTGTGCGTGGGGGGGTGAGCAGGGGG TGTGGGCGCGTCGGTCGGGCTGCAACCCCCCCTGCACCCCCCTCCCCGAGTTGCTGA GCACGGCCCGGCTTCGGGTGCGGGGCTCCGTACGGGGCGTGGCGCGGGGCTCGCCG TGCCGGGCGGGGGGTGGCGGCAGGTGGGGGTGCCGGGCGGGGCGGGGCCGCCTCG GGCCGGGGAGGGCTCGGGGGAGGGGCGCGGCGGCCCCCGGAGCGCCGGCGGCTGT CGAGGCGCGGCGAGCCGCAGCCATTGCCTTTTATGGTAATCGTGCGAGAGGGCGCA GGGACTTCCTTTGTCCCAAATCTGTGCGGAGCCGAAATCTGGGAGGCGCCGCCGCAC CCCCTCTAGCGGGCGCGGGGCGAAGCGGTGCGGCGCCGGCAGGAAGGAAATGGGC GGGGAGGGCCTTCGTGCGTCGCCGCGCCGCCGTCCCCTTCTCCCTCTCCAGCCTCGG GGCTGTCCGCGGGGGGACGGCTGCCTTCGGGGGGGACGGGGCAGGGCGGGGTTCGG CTTCTGGCGTGTGACCGGCGGCTCTAGAGCCTCTGCTAACCATGTTCATGCCTTCTTC TTTTTCCTACAGCTCCTGGGCAaCGTGCTGGTTATTGTGCTGTCTCATCATTTTGGCA AAGaattcgagctcggtacccggggatcctctagagtcgacctgcaggAATTCGAGCTCCTAGGATATCAATT GTTAATTAAGnnnAATTCACTCCTCAGGTGCAGGCTGCCTATCAGAAGGTGGTGGCTG GTGTGGCCAATGCCCTGGCTCACAAATACCACTGAGATCTTTTTCCCTCTGCCAAAA ATTATGGGGACATCATGAAGCCCCTTGAGCATCTGACTTCTGGCTAATAAAGGAAAT TTATTTTCATTGCAATAGTGTGTTGGAATTTTTTGTGTCTCTCACTCGGAAGGACATA TGGGAGGGCAAATCATTTAAAACATCAGAATGAGTATTTGGTTTAGAGTTTGGCAAC ATATGCCCATATGCTGGCTGCCATGAACAAAGGTTGGCTATAAAGAGGTCATCAGT ATATGAAACAGCCCCCTGCTGTCCATTCCTTATTCCATAGAAAAGCCTTGACTTGAG GTTAGATTTTTTTTATATTTTGTTTTGTGTTATTTTTTTCTTTAACATCCCTAAAATTTT CCTTACATGTTTTACTAGCCAGATTTTTCCTCCTCTCCTGACTACTCCCAGTCATAGC TGTCCCTCTTCTCTTATGGAGATCCCTCGACCTGCAGCCCAAGCTTATCGATACCGTC GACCTCGAGGGGGGGCCCGGTACCCAGCTTTTGTTCCCTTTGCGGCCGCAGGAACCC CTAGTGATGGAGTTGGCCACTCCCTCTCTGCGCGCTCGCTCGCTCACTGAGGCCGGG CGACCAAAGGTCGCCCGACGCCCGGGCGGCCTCAGTGAGCGAGCGAGCGCGCAGCT GCCTGCAgGACATGTGAGCAAAAGGCCAGCAAAAGGCCAGGAACCGTAAAAAGGC CGCGTTGCTGGCGTTTTTCCATAGGCTCCGCCCCCCTGACGAGCATCACAAAAATCG ACGCTCAAGTCAGAGGTGGCGAAACCCGACAGGACTATAAAGATACCAGGCGTTTC CCCCTGGAAGCTCCCTCGTGCGCTCTCCTGTTCCGACCCTGCCGCTTACCGGATACCT GTCCGCCTTTCTCCCTTCGGGAAGCGTGGCGCTTTCTCATAGCTCACGCTGTAGGTAT CTCAGTTCGGTGTAGGTCGTTCGCTCCAAGCTGGGCTGTGTGCACGAACCCCCCGTT CAGCCCGACCGCTGCGCCTTATCCGGTAACTATCGTCTTGAGTCCAACCCGGTAAGA CACGACTTATCGCCACTGGCAGCAGCCACTGGTAACAGGATTAGCAGAGCGAGGTA TGTAGGCGGTGCTACAGAGTTCTTGAAGTGGTGGCCTAACTACGGCTACACTAGAA GAACAGTATTTGGTATCTGCGCTCTGCTGAAGCCAGTTACCTTCGGAAAAAGAGTTG GTAGCTCTTGATCCGGCAAACAAACCACCGCTGGTAGCGGTGGTTTTTTTGTTTGCA AGCAGCAGATTACGCGCAGAAAAAAAGGATCTCAAGAAGATCCTTTGATCTTTTCT ACGGGGTCTGACGCTCAGTGGAACGAAAACTCACGTTAAGGGATTTTGGTCATGGG CCGCATCATGAACAATAAAACTGTCTGCTTACATAAACAGTAATACAAGGGGTGTT ATGAGCCATATTCAACGGGAAACGTCTTGCTCGAGGCCGCGATTAAATTCCAACATG GATGCTGATTTATATGGGTATAAATGGGCTCGCGATAATGTCGGGCAATCAGGTGCG ACAATCTATCGATTGTATGGGAAGCCCGATGCGCCAGAGTTGTTTCTGAAACATGGC AAAGGTAGCGTTGCCAATGATGTTACAGATGAGATGGTCAGACTAAACTGGCTGAC GGAATTTATGCCTCTTCCGACCATCAAGCATTTTATCCGTACTCCTGATGATGCATGG TTACTCACCACTGCGATCCCCGGAAAAACAGCATTCCAGGTATTAGAAGAATATCCT GATTCAGGTGAAAATATTGTTGATGCGCTGGCAGTGTTCCTGCGCCGGTTGCATTCG ATTCCTGTTTGTAATTGTCCTTTTAACAGCGATCGCGTATTTCGTCTCGCTCAGGCGC AATCACGAATGAATAACGGTTTGGTTGATGCGAGTGATTTTGATGACGAGCGTAATG GCTGGCCTGTTGAACAAGTCTGGAAAGAAATGCATAAACTTTTGCCATTCTCACCGG ATTCAGTCGTCACTCATGGTGATTTCTCACTTGATAACCTTATTTTTGACGAGGGGAA ATTAATAGGTTGTATTGATGTTGGACGAGTCGGAATCGCAGACCGATACCAGGATCT TGCCATCCTATGGAACTGCCTCGGTGAGTTTTCTCCTTCATTACAGAAACGGCTTTTT CAAAAATATGGTATTGATAATCCTGATATGAATAAATTGCAGTTTCATTTGATGCTC GATGAGTTTTTCTAATCAGAATTGGTTAATTGGTTGTAACACTGGCAGAGCATTACG CTGACTTGACGGGACGGCGGCTTTGTTGAATAAATCGAACTTTTGCTGAGTTGAAGG ATCAGATCACGCATCTTCCCGACAACGCAGACCGTTCCGTGGCAAAGCAAAAGTTC AAAATCACCAACTGGTCCACCTACAACAAAGCTCTCATCAACCGTGGCGGGGATCC TCTAGAGTCGACCTGCAGGCATGCAAGCTTCAGCGGCCCATGACATTAACCTATAAA AATAGGCGTATCACGAGGCCCTTTCGTCTCGCGCGTTTCGGTGATGACGGTGAAAAC CTCTGACACATGCAGCTCCCGGAGACGGTCACAGCTTGTCTGTAAGCGGATGCCGG GAGCAGACAAGCCCGTCAGGGCGCGTCAGCGGGTGTTGGCGGGTGTCGGGGCTGGC TTAACTATGCGGCATCAGAGCAGATTGTACTGAGAGTGCACCATAAAATTGTAAAC GTTAATATTTTGTTAAAATTCGCGTTAAATTTTTGTTAAATCAGCTCATTTTTTAACC AATAGACCGAAATCGGCAAAATCCCTTATAAATCAAAAGAATAGCCCGAGATAGAG TTGAGTGTTGTTCCAGTTTGGAACAAGAGTCCACTATTAAAGAACGTGGACTCCAAC GTCAAAGGGCGAAAAACCGTCTATCAGGGCGATGGCCCACTACGTGAACCATCACC CAAATCAAGTTTTTTGGGGTCGAGGTGCCGTAAAGCACTAAATCGGAACCCTAAAG GGAGCCCCCGATTTAGAGCTTGACGGGGAAAGCCGGCGAACGTGGCGAGAAAGGA AGGGAAGAAAGCGAAAGGAGCGGGCGCTAAGGCGCTGGCAAGTGTAGCGGTCACG CTGCGCGTAACCACCACACCCGCCGCGCTTAATGCGCCGCTACAGGGCGCGTACTAT GGTTGCTTTGACGTAtGCGGCCTTTTTACGGTTCCTGGCCTTTTGCTGGCCTTTTGCTC ACATGTC (SEQ ID NO:5).

The present disclosure also provides viral vectors comprising any of the recombinant AAV nucleic acid molecules described herein. In some embodiments, the serotype of the AAV vector is AAVrh.10. In some embodiments, the AAV vectors have restricted tropism (target cell population). In some embodiments, the AAV vectors have liver tropism.

The present disclosure also provides methods of producing a recombinant AAV vector. In some embodiments, the methods of producing a recombinant AAV vector comprise: co-transfecting a host cell with CAG-hCBS DNA surrounded by AAV ITRs and a helper nucleic acid molecule that comprises the AAV Rep and Cap sequences and adenovirus helper functions E4, E2a and VA; and culturing the host cell for a period of time sufficient to produce the recombinant AAV vector. In some embodiments, the methods of producing a recombinant AAV vector comprise: co-transfecting a host cell with CAG-hCBS DNA surrounded by AAV ITRs and two helper nucleic acid molecules, the first helper nucleic acid molecule comprising the AAV Rep and Cap sequences, and the second helper nucleic acid molecule comprising the adenovirus helper functions E4, E2a and VA; and culturing the host cell for a period of time sufficient to produce the recombinant AAV vector. In some embodiments, the methods of producing a recombinant AAV vector comprise: transfecting a host cell with CAG-hCBS DNA surrounded by AAV ITRs, wherein the host cell expresses AAV Cap and Rep proteins and adenoviral replication proteins E2, E4, and VA; and culturing the host cell for a period of time sufficient to produce the recombinant AAV vector.

In some embodiments, the methods further comprise obtaining a lysate from the cell. In some embodiments, the methods further comprise purifying the viral vector from the lysate. In some embodiments, an affinity capture method as provided herein is performed using an antibody-capture affinity resin. In some embodiments, the solid support is a cross-linked 6% agarose matrix having an average particle size of about 34 μm and having an AAV-specific antibody. An example of one such commercially available affinity resin is AVB Sepharose™ high performance affinity resin using an AAV-specific camelid-derived single chain antibody fragment of llama origin which is commercially available from GE Healthcare (AVB Sepharose). The manufacturer's literature further recommends up to a 150 cm/h flow rate and a relatively low loading salt concentration. Other suitable affinity resins may be selected or designed which contain an AAV-specific antibody, AAV1 specific antibody, or other immunoglobulin construct which is an AAV-specific ligand. Such solid supports may be any suitable polymeric matrix material, e.g., agarose, sepharose, sephadex, amongst others. Suitable loading amounts may be in the range of about 2 to about 5×10¹⁵ GC, or less, based on the capacity of a 30-mL column. Equivalent amounts may be calculated for other sized columns or other vessels.

Methods for generating and isolating AAV vectors suitable for delivery to a subject are described in, for example, U.S. Pat. Nos. 7,790,449, 7,282,199, and 7,588,772, and PCT Publication Nos. WO 2003/042397, WO 2005/033321, and WO 2006/110689. In general, production of AAV vectors involves the following components present within a single cell (denoted herein as a packaging cell): AAV genome, AAV rep and cap genes separate from (i.e., not in) the AAV genome, and helper virus functions. In some embodiments, the AAV rep and cap genes may be from any AAV serotype for which recombinant virus can be derived and may be from a different AAV serotype than the AAV genome ITRs, including, but not limited to, the AAV serotypes described herein. In some embodiments, a producer cell line is transiently transfected with a nucleic acid molecule that encodes the transgene (e.g., encoding any of the hCBS polypeptides described herein) flanked by ITRs and a nucleic acid molecule that encodes rep and cap. In some embodiments, a packaging cell line that stably supplies rep and cap is transiently transfected with a nucleic acid molecule encoding the transgene (e.g., encoding any of the hCBS polypeptides described herein) flanked by ITRs. In each of these systems, AAV vectors are produced in response to infection with helper adenovirus, E1-deleted adenovirus, or herpesvirus, requiring the separation of the AAVs from contaminating virus. More recently, systems have been developed that do not require infection with helper virus to recover the AAV; the required helper functions (i.e., adenovirus E1, E2a, VA, and E4 or herpesvirus UL5, ULB, UL52, and UL29, and herpesvirus polymerase) are supplied in trans by the system. In these newer systems, the helper functions can be supplied by transient transfection of the cells with nucleic acid molecules that encode the required helper functions, or the cells can be engineered to stably contain genes encoding the helper functions, the expression of which can be controlled at the transcriptional or posttranscriptional level. In some embodiments, the transgene (e.g., encoding any of the hCBS polypeptides described herein) flanked by ITRs and rep/cap genes are introduced into insect cells by infection with baculovirus-based vectors. Methods of making and using these and other AAV production systems are also described in U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

In some embodiments, an AAV Cap for use in the methods described herein can be generated by mutagenesis (i.e., by insertions, deletions, or substitutions) of one of the aforementioned AAV Caps or its encoding nucleic acid molecule. In some embodiments, the AAV Cap is at least about 70%, at least about 75%, at least about 80%, at least about 85%, at least about 90%, at least about 95%, at least about 96%, at least about 97%, at least about 98%, at least about 99%, or 100% identical to one or more of the aforementioned AAV Caps.

In some embodiments, an AAV Cap for use in a AAV composition is engineered to contain a heterologous sequence or other modification. For example, a peptide or protein sequence that confers selective targeting or immune evasion may be engineered into a Cap protein. Alternately or in addition, the Cap may be chemically modified so that the surface of the AAV is polyethylene glycolated (i.e., pegylated), which may facilitate immune evasion. The Cap protein may also be mutagenized (e.g., to remove its natural receptor binding, or to mask an immunogenic epitope).

The present disclosure also provides methods of generating a packaging cell comprising creating a cell line that stably expresses all the necessary components for AAV viral particle production. For example, a plasmid (or multiple plasmids) comprising a AAV genome lacking AAV rep and cap genes, AAV rep and cap genes separate from the AAV genome, and a selectable marker, such as a neomycin resistance gene, are integrated into the genome of a cell. AAV genomes have been introduced into bacterial plasmids by procedures such as GC tailing (Samulski et al., Proc. Natl. Acad., 1982, 79, 2077-2081), addition of synthetic linkers containing restriction endonuclease cleavage sites (Laughlin et al., Gene, 1983, 23, 65-73) or by direct, blunt-end ligation (Senapathy & Carter, J. Biol. Chem., 1984, 259, 4661-4666). The packaging cell line is then infected with a helper virus such as adenovirus. The advantages of this method are that the cells are selectable and are suitable for large-scale production of AAV. Other examples of suitable methods employ adenovirus or baculovirus rather than plasmids to introduce AAV genomes and/or rep and cap genes into packaging cells.

In some embodiments, the AAV may be prepared as described in, for example, U.S. Patent Application Publication No. 2009/0275107, which provides an optionally continuous process for producing AAV and isolating from cell culture without requiring cell permeabilization and/or cell lysis. Alternately, AAVrh.10-based AAV vectors or AAV with engineered capsids as described herein may be purified using the methods described herein.

In some embodiments, packaging cells that produce infectious AAV are also provided. In some embodiments, the packaging cells are any cell lines that provide adenovirus helper functions required for the transfection method. In some embodiments, packaging cells may be stably transformed cancer cells such as HeLa cells, HEK 293 cells, HEK 293T cells, PerC.6 cells (a cognate 293 line), and SF9 cells, or any other insect or mammalian cell lines transduced to express helper functions. In some embodiments, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

In some embodiments, the AAV may be purified by methods standard in the art such as by column chromatography or cesium chloride gradients. Methods for purifying AAV vectors from helper virus are known in the art (see, Clark et al., Hum. Gene Ther., 1999, 10, 1031-1039; Schenpp and Clark, Methods Mol. Med., 2002, 69, 427-443; U.S. Pat. No. 6,566,118; and PCT Publication No. WO 98/09657).

Methods of altering the AAV vector may involve a variety of techniques, which techniques are known to those of skill in the art. For example, site directed mutagenesis may be performed at the level of the nucleic acids encoding one or more amino acids to be altered. Alternately, an insertion of one or more amino acids (e.g., 2, 3, 4, 5 or more) may be made at the target region within the AAV capsid.

Additional expression systems can also be used. For example, a baculovirus/insect cell (e.g., Sf9) expression system can be used. Examples of such expression vectors and insect cell expression systems and methods are described in The Baculovirus Expression System: A Laboratory Guide, Linda King, Springer; 2012. Baculovirus promoters such as baculovirus polyhedrin and p10 promoters are commercially available (see, e.g., Invitrogen's “Guide to Baculovirus Expression Vector Systems (BEVS) and Insect Cell Culture Techniques”, 2002 (Life Technologies, Carlsbad, Calif.) and F. J. Haines et al. “Baculovirus Expression Vectors”, undated (Oxford Expression Technologies, Oxford, UK).

The engineered AAV may also be generated using methods described herein, or other methods described in the art, and purified as described (see, Mietzsch et al, Hum. Gene Ther., 2014, 25, 212-222; Smith et al, Mol. Ther., 2009, 17, 1888-96), describing a simplified baculovirus-AAV vector expression system coupled with one-step affinity purification. For example, lystates or supernatants (e.g., treated, freeze-thaw supernatants or media containing secreted AAV), may be purified using one-step AVB sepharose affinity chromatography using 1 ml prepacked HiTrap columns on an ACTA purifier (GE Healthcare) as described by manufacturer.

In some embodiments, the methods of producing AAV vectors comprise using host cells that are mammalian cells. In these embodiments, the mammalian cell can be HEK 293 cell, HEK 293T cell, PerC.6 cell, or any other cell line comprising Adenovirus E1 helper function. In these embodiments, the CAG-hCBS DNA surrounded by AAV ITRs is in pAAV-CAG-hCBS plasmid and the helper nucleic acid molecule is a helper plasmid. Suitable examples of helper plasmids include, but are not limited to, pPAKMArh.10; pXX6-80 (Aldevron); pRepCap, pAdDeltaF6, pAAV-syn, and pDGM (Addgene); pGMAAV (Genmedi); pRepCap and pHELP (Applied Viromnics); pDP (Karolinska Institute); as well as helper plasmids described in, for example, U.S. Patent Application Publication No. 2004/0235174, PCT Publication No. WO 02/012525, EP 1983057, and U.S. Pat. No. 6,846,665. In some embodiments, the helper plasmid is pPAKMArh.10.

In some embodiments, the methods of producing AAV vectors comprise using host cells that are insect cells. In these embodiments, the insect cells can be Sf9 cells. In these embodiments, the CAG-hCBS DNA surrounded by AAV ITRs is present in a baculovirus or a Bacmid.

The present disclosure also provides compositions comprising any of the AAV nucleic acid molecules or AAV vectors described herein. In some embodiments, the compositions comprise the AAV nucleic acid molecules or AAV vectors and a pharmaceutically acceptable carrier. The compositions may also comprise other ingredients such as diluents. Acceptable carriers and diluents are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counter ions such as sodium; and/or nonionic surfactants such as Tween, pluronics or polyethylene glycol (PEG).

Titers of AAV vectors in the compositions will vary depending, for example, on the particular AAV vector, the mode of administration, the treatment goal, the individual being treated, and the cell type(s) being targeted, and may be determined by methods standard in the art. Titers of AAV vectors may be about 1×10⁶, about 1×10⁷, about 1×10⁸, about 1×10⁹, about 1×10¹⁰, about 1×10¹¹, about 1×10¹², about 1×10¹³, or about 1×10¹⁴ or more particles per ml, such as DNase resistant particles (DRP) per ml.

In some embodiments, quantification of the genome copies (“GC”) may be used as the measure of the dose contained in the compositions. Any method known in the art can be used to determine the GC number. One method for performing AAV GC number titration is as follows: purified AAV vector samples are first treated with DNase to eliminate un-encapsidated AAV genome DNA or contaminating plasmid DNA from the production process. The DNase resistant particles are then subjected to heat treatment to release the genome from the capsid. The released genomes are then quantitated by real-time PCR using primer/probe sets targeting specific region of the viral genome (usually poly A signal). The AAV vectors can be formulated in dosage units to contain an amount of AAV vector that is in the range of about 1.0×10⁷ GC to about 1.0×10¹⁵ GC (to treat an average subject of 70 kg in body weight), and preferably 1.0×10¹² GC to 1.0×10¹⁴ GC for a human patient. In some embodiments, the dose is 1.0×10⁷ GC, 5.0×10⁷ GC, 1.0×10⁸ GC, 5.0×10⁸ GC, 1.0×10⁹ GC, 5.0×10⁹ GC, 1.0×10¹⁰ GC, 5.0×10¹⁰ GC, 1.0×10¹¹ GC, 5.0×10¹¹ GC, 1.0×10¹² GC, 5.0×10¹² GC, 1.0×10¹³ GC, 5.0×10¹³ GC, 1.0×10¹⁴ GC, 5.0×10¹⁴ GC, or 1.0×10¹⁵ GC. In some embodiments, dosages may also be expressed in units of GC per kilogram (kg) of bodyweight (i.e., 1×10¹⁰ GC/kg, 1×10¹¹ GC/kg, 1×10¹² GC/kg, 1×10¹³ GC/kg, 1×10¹⁴ GC/kg, 1×10¹⁵ GC/kg, respectively). Methods for titering AAV are described in Clark et al., Hum. Gene Ther., 1999, 10, 1031-1039.

The present disclosure also provides methods of preventing or treating a disease, disorder, or condition associated with elevated homocysteine in a subject in need thereof, comprising administering to the subject any of the AAV nucleic acid molecules encapsidated within any of the AAV vectors, or compositions comprising the same, described herein. In some embodiments, the disease, disorder, or condition associated with elevated homocysteine is CBS deficiency. In some embodiments, the disease, disorder, or condition associated with elevated homocysteine is prevented. In some embodiments, the disease, disorder, or condition associated with elevated homocysteine is treated.

In some embodiments, the disease, disorder, or condition associated with elevated homocysteine is associated with particular genes, such as, for example, methylmalonic aciduria and homocystinuria type C (MMACHC (cb1C)), methylmalonic aciduria and homocystinuria type D (MMADHC (cb1D-combined and cb1D-homocystinuria)), 5-methyltetrahydrofolate-homocysteine methyltransferase reductase (MTRR(cblE)), LMBR1 domain containing 1 (LMBRD1 (cb1F)), 5-methyltetrahydrofolate-homocysteine methyltransferase (MTR (cb1G)), ATP binding cassette subfamily D member 4 (ABCD4 (cb1J)), THAP domain containing 11 (THAP11(cb1X-like)), zinc finger protein 143 (ZNF143(cb1X-like)), or a hemizygous variant in host cell factor C1 (HCFC1 (cb1X)). In some embodiments, the disease, disorder, or condition associated with elevated homocysteine is methylenetetrahydrofolate reductase (MTHFR) deficiency. In some embodiments, the disease, disorder, or condition associated with elevated homocysteine is low folate, low B6, or low B12 status. In some embodiments, the disease, disorder, or condition associated with elevated homocysteine is idiopathic hyperhomocystaneimia. In some embodiments, the disease, disorder, or condition associated with elevated homocysteine is in individuals with only slightly elevated tHcy, which may include atherosclerosis, thrombosis, and osteoporosis.

The present disclosure also provides methods of transducing or transfecting a target cell with AAV nucleic acid molecules encapsidated within AAV vectors, or compositions comprising the same, described herein in vivo or in vitro. The in vivo methods comprise the step of administering an effective dose, or effective multiple doses, of a composition comprising an AAV nucleic acid molecule encapsidated within an AAV vector, or composition comprising the same, described herein to an animal (including a human) in need thereof. If the dose is administered prior to development of a disorder/disease, the administration is prophylactic. If the dose is administered after the development of a disorder/disease, the administration is therapeutic. In some embodiments, an effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival.

In some embodiments, combination therapies are provided. Combination treatments includes both simultaneous treatment and sequential treatments. Combination treatments include any of the methods described herein and standard medical treatments (e.g., anethole, dithiolethione, or betaine). In some embodiments, any of the methods described herein can be used in combination with lifestyle changes, such as relaxation of a protein restricted diet.

Administration of an effective dose of the compositions may be by routes standard in the art including, but not limited to, intramuscular, parenteral, intravenous, oral, buccal, nasal, pulmonary, intracranial, intraosseous, intraocular, rectal, or vaginal. Systemic administration is administration into the circulatory system so that the entire body is affected. Systemic administration includes enteral administration such as absorption through the gastrointestinal tract and parental administration through injection, infusion or implantation. In some embodiments, the composition is administered to the subject by intramuscular injection or intravenous injection.

Administration of an AAV nucleic acid molecule encapsidated within an AAV vector, or composition comprising the same, described herein may also be accomplished by using any physical method that will transport the AAV vector into the target tissue of an animal. Administration includes, but is not limited to, injection into muscle, the bloodstream and/or directly into the liver. In some embodiments, the compositions can be prepared as injectable formulations or as topical formulations to be delivered to the muscles by transdermal transport. Numerous formulations for both intramuscular injection and transdermal transport have been previously developed and can be used in the practice of the methods described herein.

For purposes of injection, sterile aqueous solutions can be employed. Such aqueous solutions can be buffered, if desired, and the liquid diluent first rendered isotonic with saline or glucose. Solutions of an AAV vector as a free acid or a pharmacologically acceptable salt can be prepared in water suitably mixed with a surfactant such as hydroxpropylcellulose. A dispersion of an AAV vector can also be prepared in glycerol, liquid polyethylene glycols and mixtures thereof and in oils. Under ordinary conditions of storage and use, these preparations can contain a preservative to prevent the growth of microorganisms.

In some embodiments, the pharmaceutical carriers, diluents or excipients suitable for injectable use include sterile aqueous solutions or dispersions and sterile powders for the extemporaneous preparation of sterile injectable solutions or dispersions. In some embodiments, the form is sterile and fluid to the extent that easy syringability exists. In some embodiments, the carrier can be a solvent or dispersion medium containing, for example, water, ethanol, polyol (for example, glycerol, propylene glycol, liquid polyethylene glycol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

In some embodiments, sterile injectable solutions are prepared by incorporating an AAV vector in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the methods of preparation are vacuum drying and the freeze drying technique that yield a powder of the active ingredient plus any additional desired ingredient from the previously sterile-filtered solution thereof.

In some embodiments, transduction or transfection of cells with an AAV nucleic acid molecule encapsidated within an AAV vector may also be carried out in vitro. In some embodiments, desired target muscle cells are removed from the subject, transduced with an AAV nucleic acid molecule encapsidated within an AAV vector and reintroduced into the subject. Alternately, syngeneic or xenogeneic muscle cells can be used where those cells will not generate an inappropriate immune response in the subject.

Suitable methods for the transduction and reintroduction of transduced cells into a subject are known in the art. In some embodiments, cells can be transduced in vitro by combining an AAV nucleic acid molecule encapsidated within an AAV vector with muscle cells, e.g., in appropriate media, and screening for those cells harboring the DNA of interest using conventional techniques such as Southern blots and/or PCR, or by using selectable markers. Transduced cells can then be formulated into pharmaceutical compositions, and the composition introduced into the subject by various techniques, such as by intramuscular, intravenous, subcutaneous and intraperitoneal injection, or by injection into smooth and cardiac muscle, by using, for example, a catheter.

Transduction of cells with the AAV nucleic acid molecules encapsidated within AAV vectors described herein results in sustained expression of CBS. In some embodiments, the AAV-borne CBS transgene is efficiently expressed in transduced cells. Methods to measure protein expression levels of CBS include, but are not limited to, Coomasie blue or silver staining of protein in a separation media, such as gel electrophoresis, Western blotting, immunocytochemistry, other immunologic-based assays; assays based on a property of the protein including but not limited to, enzyme assays, ligand binding or interaction with other protein partners. Binding assays are also well known in the art. For example, a BlAcore instrument can be used to determine the binding constant of a complex between two proteins. The dissociation constant for the complex can be determined by monitoring changes in the refractive index with respect to time as buffer is passed over the chip. Other suitable assays for measuring the binding of one protein to another include, for example, immunoassays such as enzyme linked immunoabsorbent assays (ELISA) and radioimmunoassays (MA), or determination of binding by monitoring the change in the spectroscopic or optical properties of the proteins through fluorescence, UV absorption, circular dichrosim, or nuclear magnetic resonance (NMR).

The present disclosure also provides methods of regulating biological processes, including cystathionine production, by regulating the expression and/or activity of CBS. In some embodiments, the methods regulate cystathionine production in an animal or human patient, wherein the patient is protected from or treated for a disease that is amenable to regulation of cystathionine production, such as homocystinuria and conditions/symptoms related thereto (e.g., dislocated optic lenses, skeletal disorders, mental retardation and premature arteriosclerosis and thrombosis). As used herein, the phrase “protected from a disease” refers to reducing the symptoms of the disease; reducing the occurrence of the disease, and/or reducing the severity of the disease. Protecting a patient can refer to the ability of a therapeutic composition, when administered to a patient, to prevent a disease from occurring and/or to cure or to treat the disease by alleviating disease symptoms, signs or causes. As such, to protect a patient from a disease includes both preventing disease occurrence (prophylactic treatment) and treating a patient that has a disease or that is experiencing initial symptoms or later stage symptoms of a disease (therapeutic treatment). More specifically, a therapeutic composition as described herein, when administered to a patient by the methods described herein, preferably produces a result which can include alleviation of the disease (e.g., reduction of at least one symptom or clinical manifestation of the disease), elimination of the disease, alleviation of a secondary disease resulting from the occurrence of a primary disease, or prevention of the disease. In some embodiments, administration of the therapeutic composition can produce a result that can include increased accumulation of downstream metabolites of transsulfuration in a mammal.

The present disclosure also provides uses of any of the compositions described herein for the preparation of a medicament for the prevention or treatment of a disease, disorder, or condition associated with elevated homocysteine in a human subject.

The present disclosure also provides uses of any of the compositions described herein for the prevention or treatment of a disease, disorder, or condition associated with elevated homocysteine in a human subject.

In order that the subject matter disclosed herein may be more efficiently understood, examples are provided below. It should be understood that these examples are for illustrative purposes only and are not to be construed as limiting the claimed subject matter in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1

### Materials and Methods

pAAVrh.10-CAG-hCBS Plasmid Construction:

The hCBS coding sequence from pUC:ΔHCBS (see, Kruger et al., Proc. Natl. Acad. Sci. USA, 1994, 91, 6614-6618) was PCR amplified using the primers 5′-CAGTCTCGAAC TTAACATGCCTTCTGAGACCCCC-3′ (SEQ ID NO:3) and 5′-GGGCCCATTACCGAT ACTTCACTTCTGGTCCGCTCC-3′ (SEQ ID NO:4), digested with AF1II and EcoRV-HF and cloned into the vector backbone of pAAV-TBG-FFLUC digested with BstAP 1 and EcoRV-HF via blunt end ligation. Because the orientation of the clone could not be obtained in the correct direction, a subsequent cloning was performed in which the hCBS insert was removed with MseI an XhoI and cloned into pAAV-CAG-MCS digested with MfeI and SacI via blunt end ligation. The final plasmid, pAAV-CAG-hCBS, was verified by sequencing.

The product pAAV-CAG-hCBS can be produced by: 1) using the baculovirus system which uses the insect virus to transduce insect cells with all of the components used to generate the AAV; 2) from a cell line that has been genetically modified to produce AAV-CAG-hCBS; and 3) from a herpes virus system that infect either proviral cell lines that contain an integrated rAAV genome or cells transfected with an rAAV plasmid or infected with rAAV.

pAAV1-CMV-hCBS Plasmid Construction:

pAAV1-CMV-hCBS was constructed by inserting a NotI fragment containing the hCBS cDNA fragment obtained from the 15ACY6PP plasmid (Invitrogen), which contains a codon optimized CBS gene. The resulting fragment was then cloned into NotI site of the pAAV-GFP vector (Addgene) containing CMV promoter and the AAV inverted terminal repeat sequences (ITR). pAAV1-CMV-hCBS was verified by sequencing.

### AAV Viral Production:

AAVrh.10-CAG-hCBS was produced essentially as described in Rosenberg et al. (Human Gene Therapy Clin. Devel., 2014, 25, 164-177). In brief, HEK 293 cells were co-transfected with pAAV-CAG-hCBS and pPAKMA.rh10, which is a helper and packaging plasmid that provides the AAV Rep proteins derived from AAV2 needed for vector replication, the AAVrh.10 viral structural (Cap) proteins, and the Ad helper functions of E2, E4, Va RNA. After 72 hours, cells were harvested and a crude viral lysate was prepared by four freeze/thaw cycles and clarified by centrifugation. AAVrh.10-CAG-hCBS was purified by iodixanol gradient and QHP anion exchange chromatography, and concentrated using an Amicon Ultra-15 100K centrifugal filter device. Vector genome titers were determined by quantitative PCR.

### Mouse Model:

Tg-I278T Cbs^{-/-} mice were generated as previously described (see, Wang et al., Hum. Mol. Genet., 2005, 14, 2201-2208). In brief male Tg-I278T Cbs^{-/-} mice were mated with female Tg-I278T Cbs^{+/-} mice in cages with water bottles containing 25 mM ZnSO₄. All pups were genotyped between 10 and 14 days of age. At the time of weaning (around 30 days), mice were put in new cages with non-zinc water. The average age of the mice in this study was 310 days (range 288-365), and an equal number of male (n=10) and females (n=10) were used. Serum was collected from animals by retro-orbital bleed.

### Gene Delivery and Experimental Schemes:

Before injection (D-7), all mice had serum collected for baseline tHcy and methionine determination. AAVrh.10-CAG-hCBS was delivered into Tg-I278T Cbs^{-/-} mice (n=16) via a single lateral tail vein injection (IV) or intraperitoneal injection (IP) at doses of 1.1×10¹⁰ (n=2), 5.6×10¹⁰ (n=2), 1.1×10¹¹ (n=2), and 5.6×10¹¹ (n=10) genome copies/mouse. Saline (150 μl) was injected for control mice. Serum was prepared from blood collected at D7, D14, D21, D28, D42, D63 and D78 after injection. All mice were euthanized by isoflurane overdose at either 21 or 78 days after injection. Liver, kidney, and serum were collected at this time.

### Immunoblots:

Tissue homogenates from liver were prepared in 10 mM Tris-HCl (pH 7.5) supplemented with protease inhibitors (Roche). 25 μg of lysate was separated by 4-12% SDS-PAGE (Invitrogen) under reducing conditions and transferred to nitrocellulose. Blots were probed with HA antibody (H9658, Sigma) for endogenous I278T CBS protein, rabbit anti-CBS sera for total CBS protein (see, Kruger, Proc. Natl. Acad. Sci. USA, 1994, 91, 6614-6618), and actin antibody (A5441, Sigma) for loading control. Gel images were captured and quantified using the FluorChem SP system (Alpha Innotech).

Measurement of Serum tHcy and Tissue CBS Activity

CBS activity in the liver and kidney was measured using a Biochrom 30 amino acid analyzer (Cambridge, UK) as described previously (see, Wang et al., Hum. Mol. Genet., 2005, 14, 2201-2208). One unit of activity is defined as nmoles of cystathionine formed/mg of tissue lysate protein/hour. Serum and tissue tHcy (a sum total of free and disulfide-bonded homocysteine) and methionine levels were measure using the Biochrom 30 amino acid analyzer as performed previously (see, Gupta et al., FASEB J., 2009, 23, 883-893).

### Statistical Analysis:

Values in text are mean±SEM. Differences between two groups were analyzed by the Mann-Whitney U test (unpaired and non-parametric test). Significance between more than two groups was determined using one-way ANOVA followed by Tukey's multiple comparison tests employing GraphPad Prism 6.0 software. Statistical significance was accepted at the value of P<0.05.

pAAV1-CMV-hCBS Plasmid Construction and Viral Production:

An hCBS cDNA fragment was obtained from 15ACY6PP plasmid (Invitrogen) by NotI digestion, and cloned into the NotI site of the pAAV-GFP vector (Addgene) containing the CMV promoter and the AAV inverted terminal repeat sequences (ITR). pAAV1-CMV-hCBS was verified by sequencing.

### Gene Delivery and Experimental Schemes:

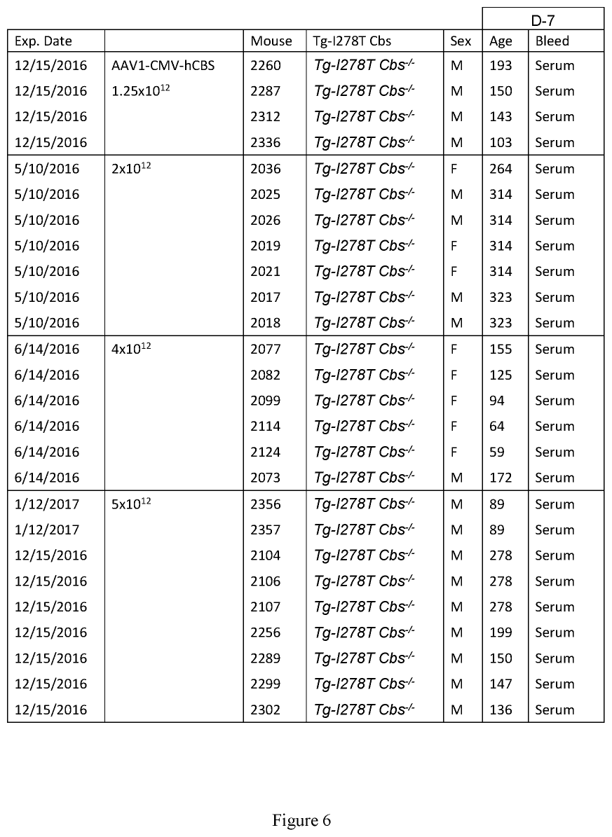

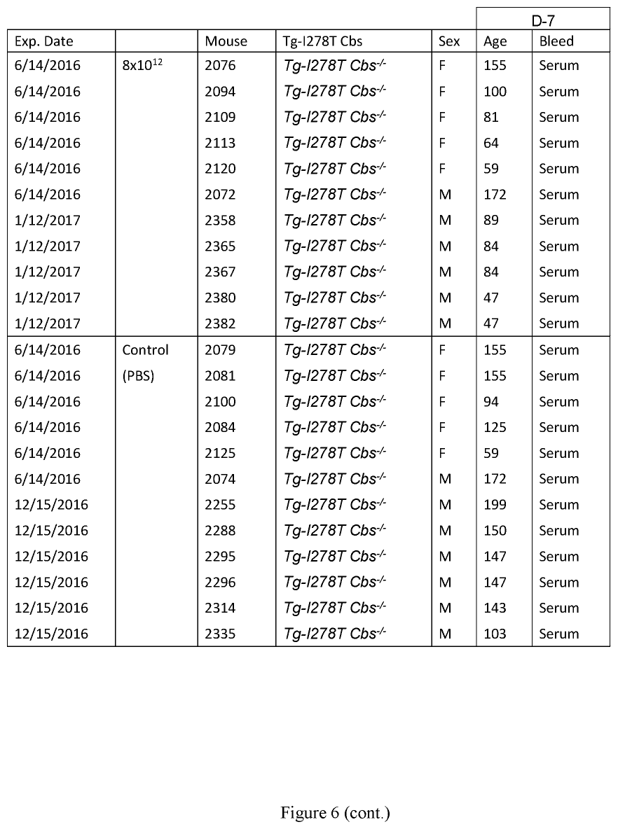

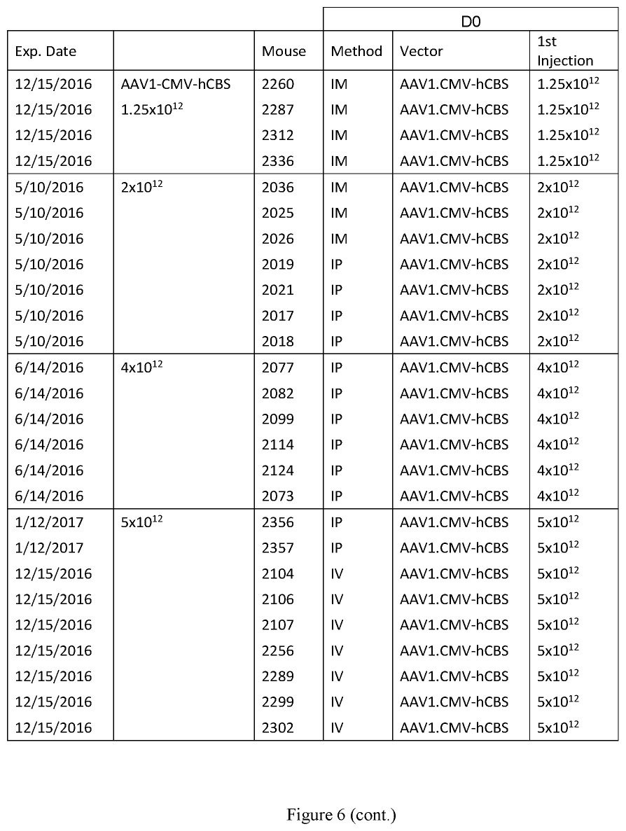

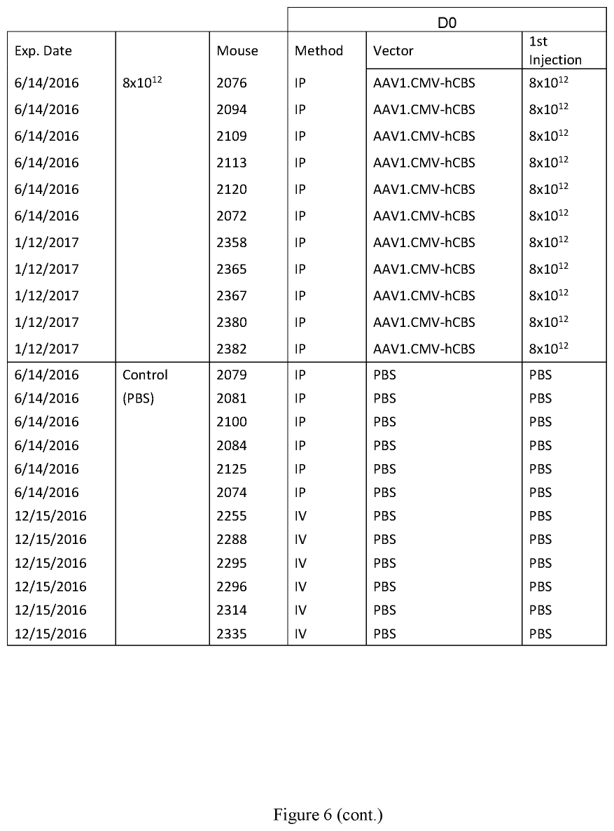

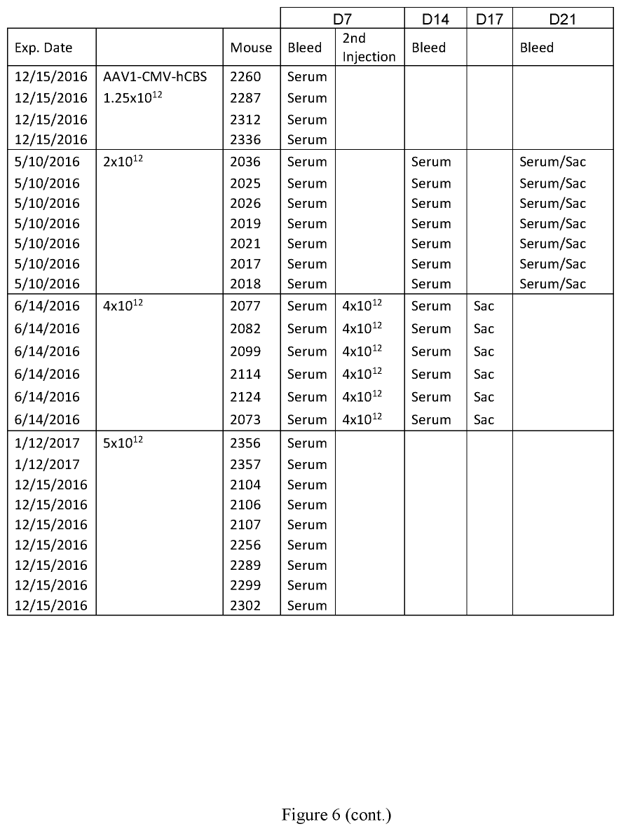

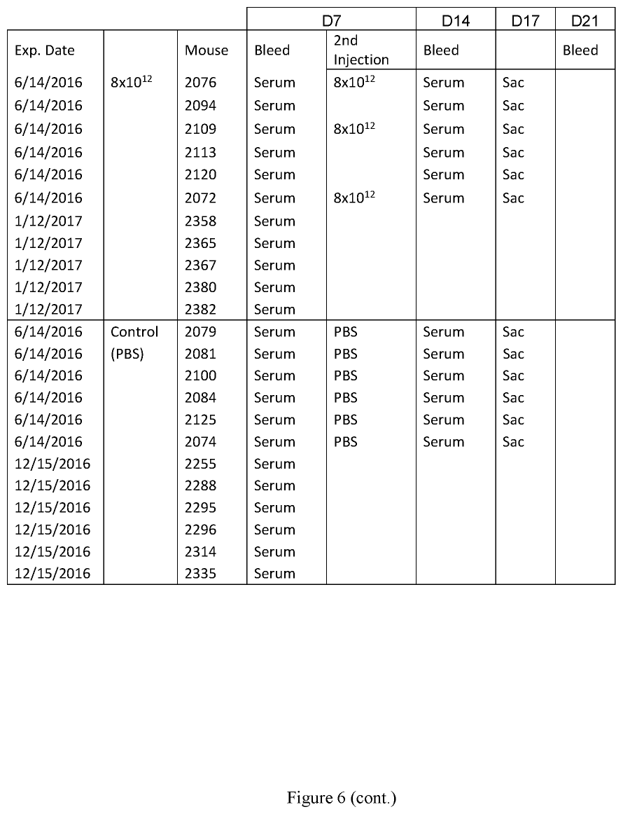

AAV1-CMV-hCBS was delivered into Tg-I278T Cbs^{-/-} mice (n=49) via a single lateral tail vein injection (IV), intraperitoneal injection (IP), or intramuscular injection (IM) at variable dosages of vector number (see, FIG. 6). Serum was prepared from blood collected before (D-7) and weekly after injection for tHcy and methionine determination. All mice were euthanized by isoflurane overdose at the end of experiments. Measurement of serum tHcy/Met, Western blot analysis, and tissue CBS activity were performed as previously described herein.

### Example 2

### Animal Model Studies

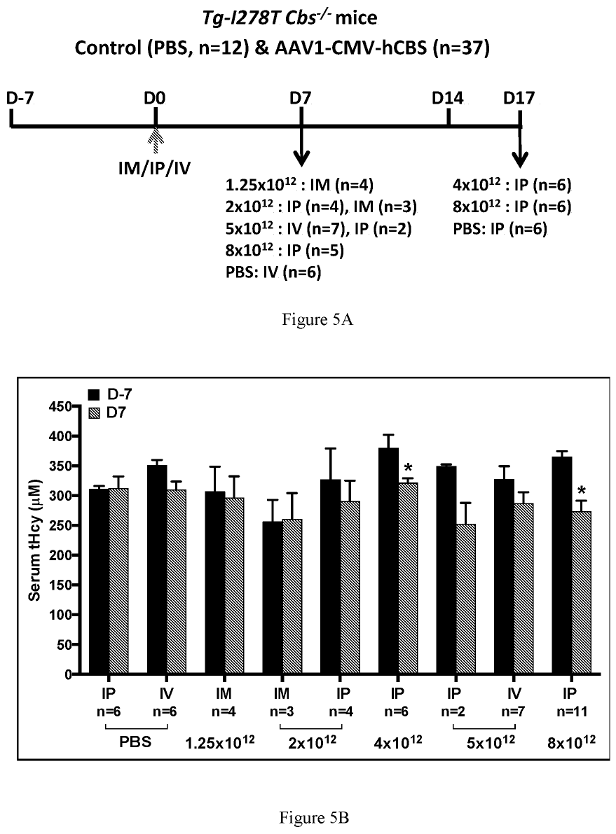

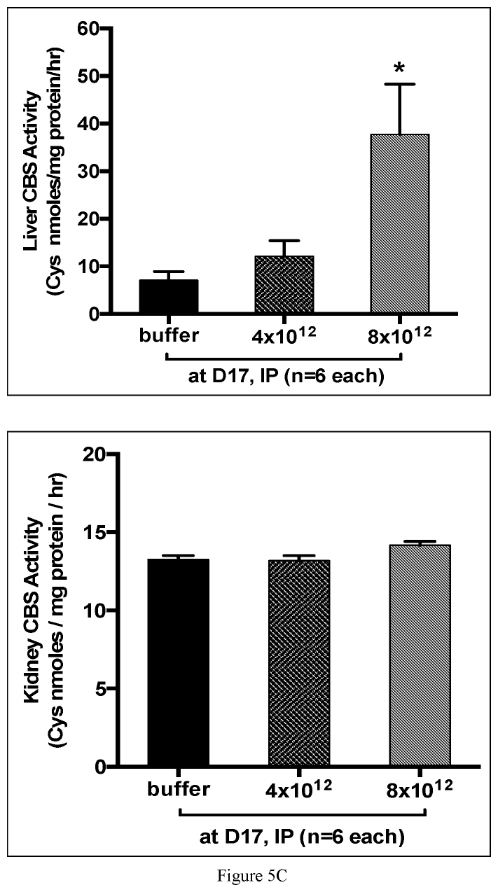

In an initial study, an rAAV vector was constructed in which the CMV promoter was placed upstream of the hCBS cDNA. This construct was then packaged in a cell line that produced AAV1 serotype viral particles (rAAV1-CMV-hCBS). Forty-nine Tg-I278T Cbs^{-/-} mice were injected with either rAAV1-CMV-hCBS (n=37) or PBS (n=12; see FIG. 5A). Mice were injected with four different doses of virus (1.25−8×10¹² genomes/mouse) using three different routes; intramuscular injection (IM), intraperitoneal injection (IP), or intravascular injection (IV). A modest but statistically significant lowering of tHcy was observed in the IP injected animals treated with either the 4×10¹² or 8×10¹² dose (see, FIG. 5B). A small but significant increase in liver CBS activity was also observed (see, FIG. 5C).

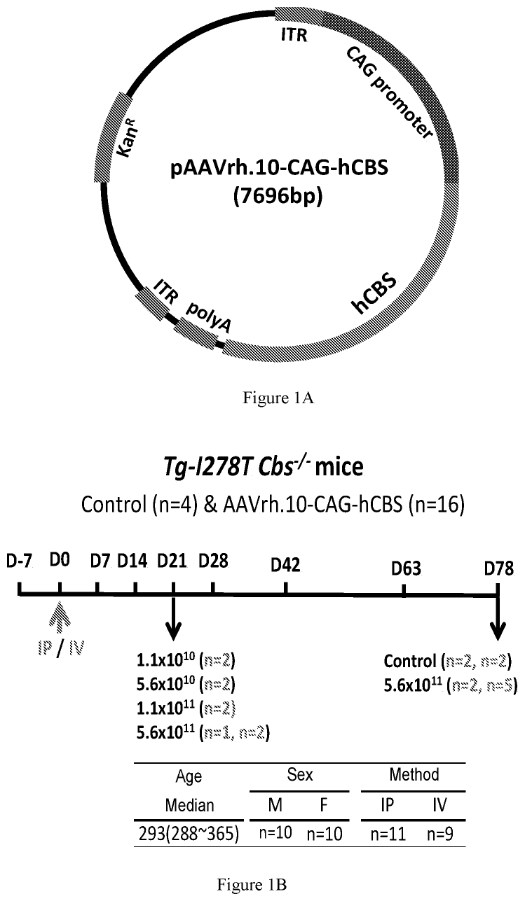

Another study was carried out using a modified vector (see, FIG. 1A). The promoter in the modified vector was changed from CMV to the CAG promoter. The CAG promoter is a hybrid promoter consisting of the CMV enhancer fused to the b-actin promoter, and has extremely high expression in liver (Nguyen et al., J. Surg. Res., 2008, 148, 60-66; and Sen et al., Scientific Reports, 2013, 3, 1832). In addition, the serotype of the virus was changed from AAV1 to AAVrh.10.

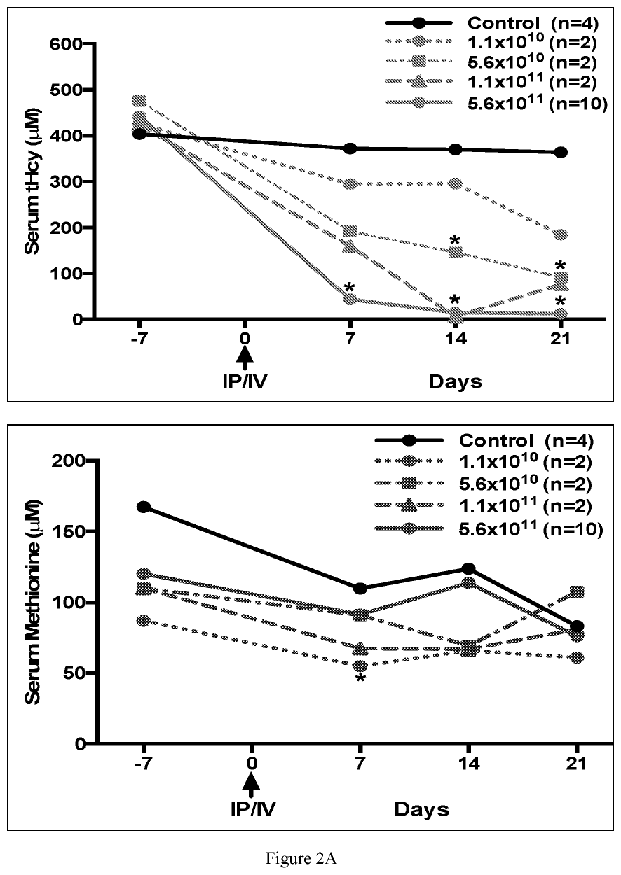

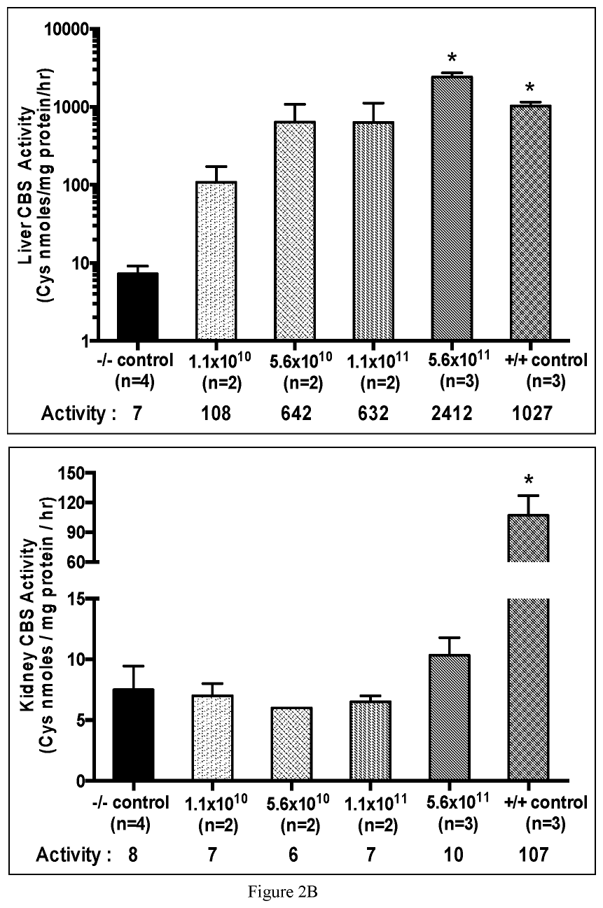

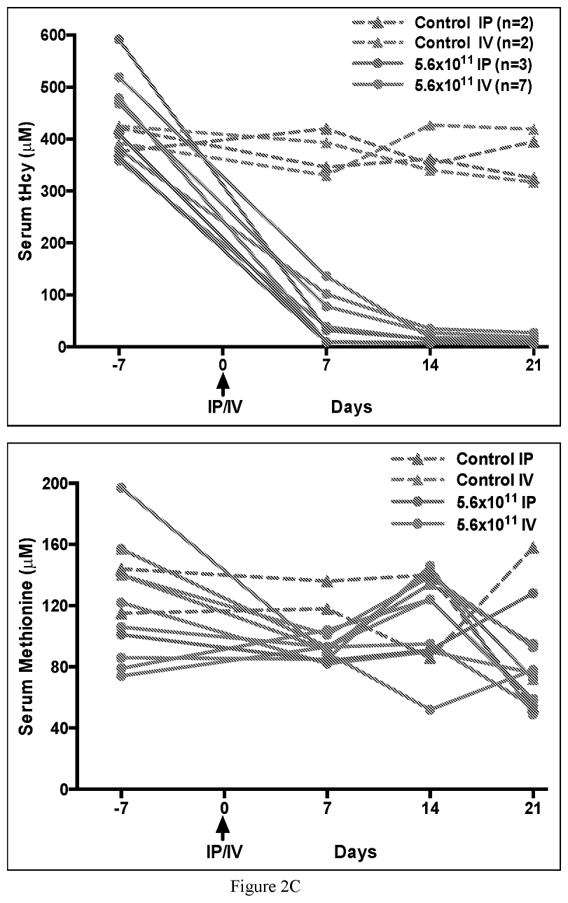

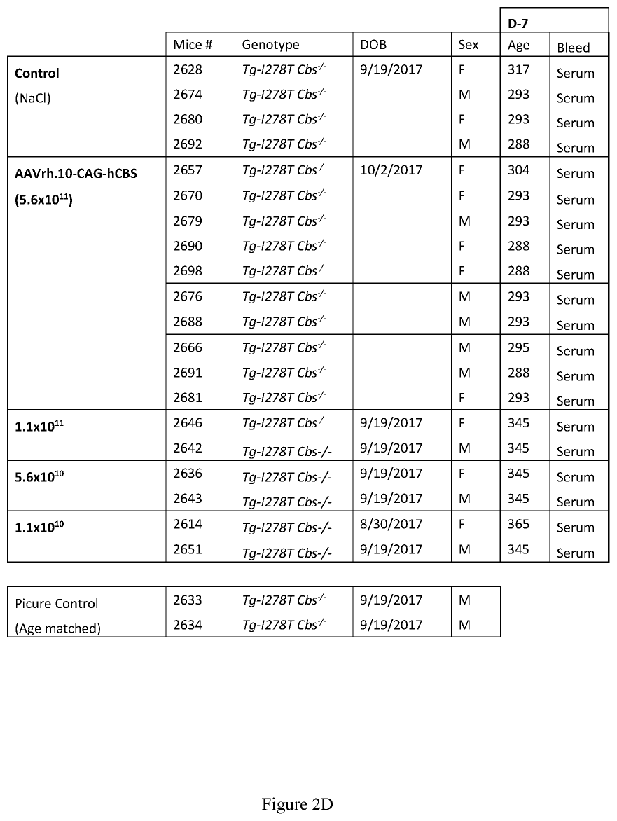

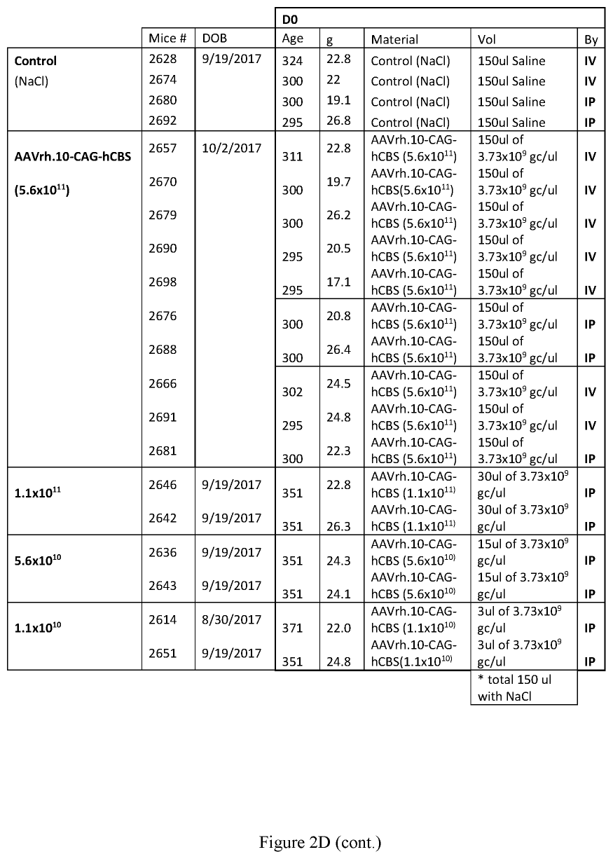

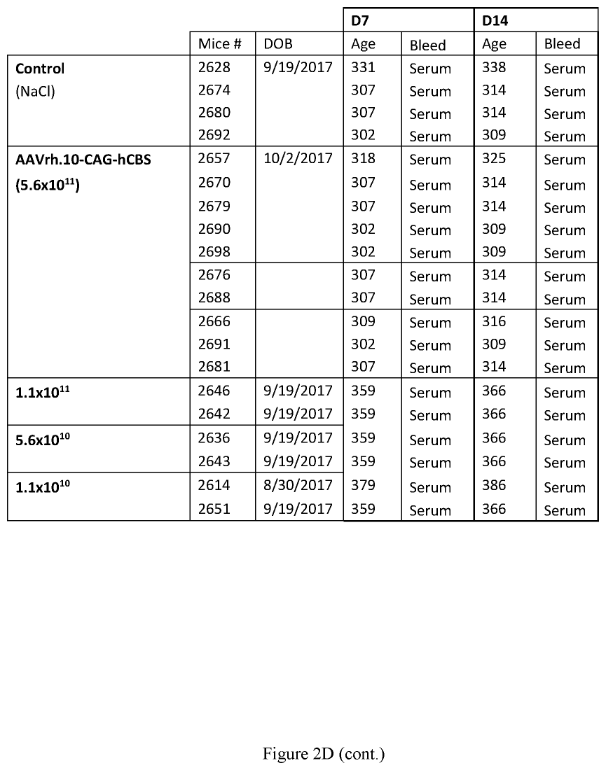

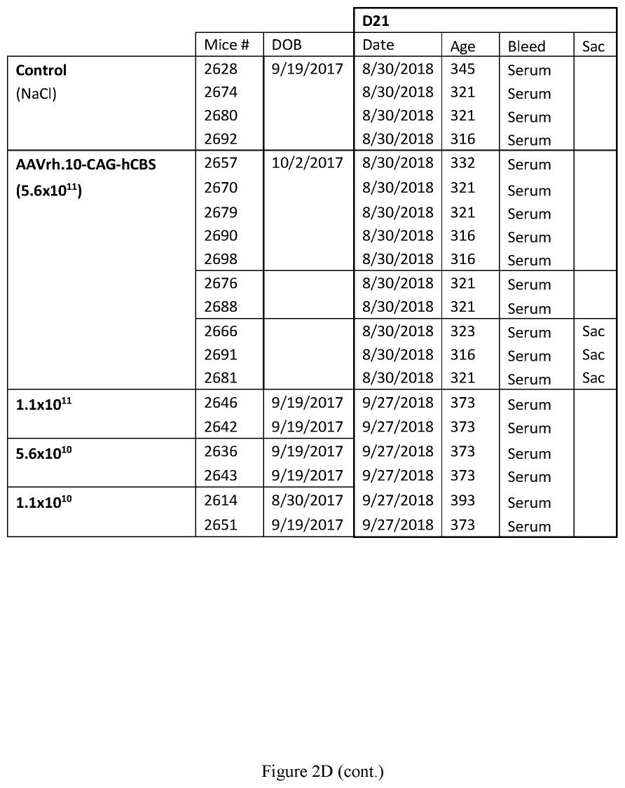

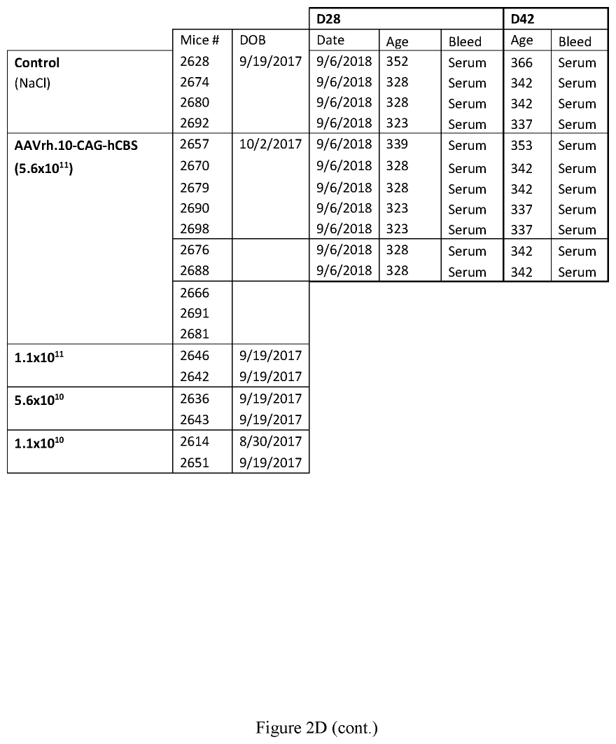

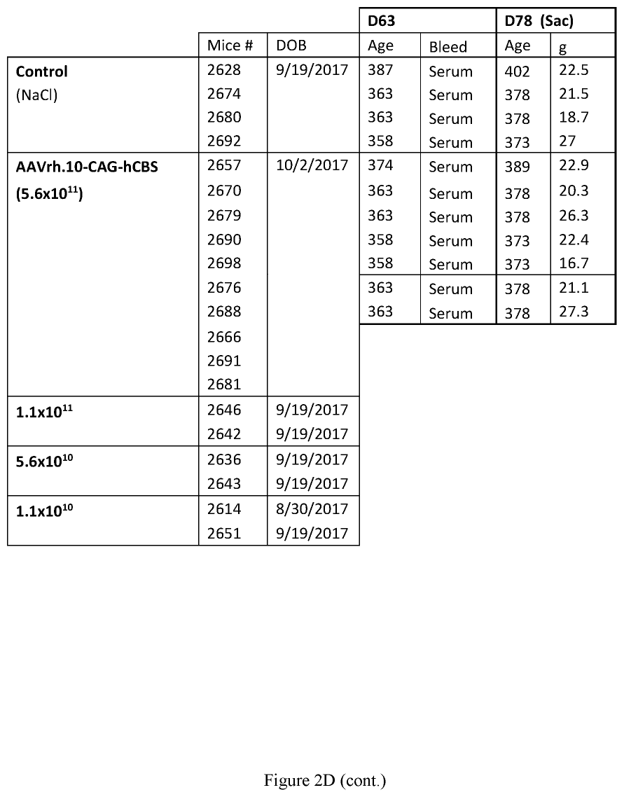

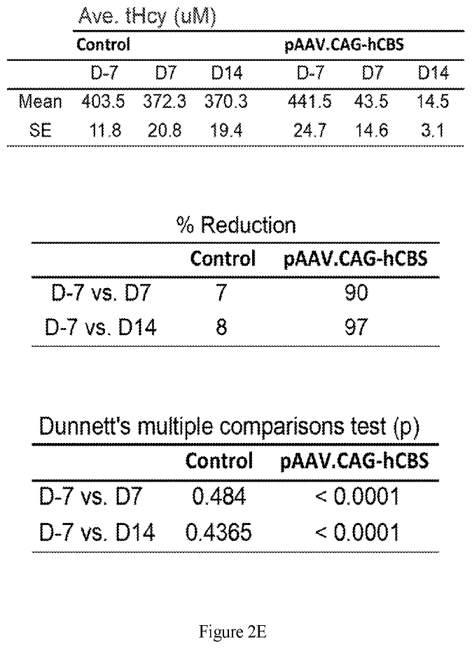

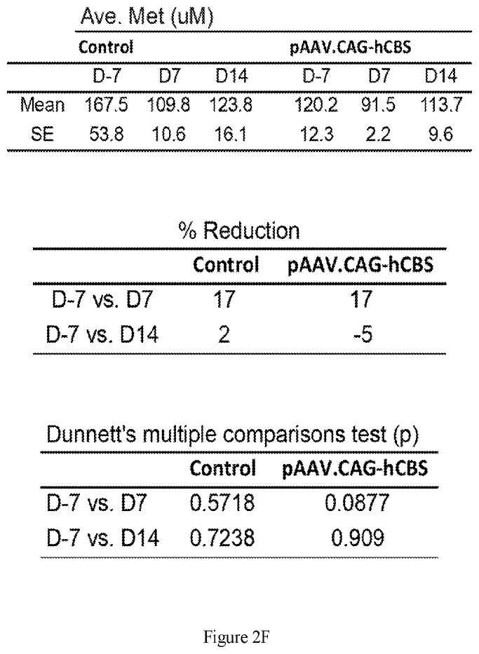

This modified vector was used to produce virus that was injected into a total of 16 Tg-I278T Cbs^{-/-} mice either IV or IP at doses ranging from 1.1×10¹⁰ to 5.6×10¹¹ genomes/mouse (see, FIG. 1B). In addition, four control animals were mock injected. Over the course of the next 21 days a highly significant decrease in mean tHcy was observed that was dosage dependent (see, FIG. 2A). Mice injected with the highest dose (5.6×10¹¹ genomes/mouse) showed a 97% decrease in tHcy after 14 days. This tHcy level was similar to what was observed in wild-type C57BL6 mice (Esse et al., FASEB J., 2014, 28, 2686-2695). Methionine was not significantly lowered by the treatment, but it should be noted that adult CBS deficient mice, unlike humans, do not show significant hypermethionemia. After 21 days, nine of the mice were euthanized and their livers were assessed for CBS activity. Significant levels of CBS activity were observed in all the treated mice, with the highest amounts of activity observed in the livers of animals treated with the highest dose of virus. In these animals, the amount of activity observed was similar to that observed in wildtype C57BL6 mice. A significant increase in kidney CBS activity was not observed, suggesting that either the AAVrh.10 serotype has poor tropism toward kidney, or the CAG promoter did not function well in kidney. No significant difference has been observed between the IP or IV routes of delivery (see, FIG. 2C). FIG. 2D shows a summary of individual mice treatment for experiments in FIGS. 2C.

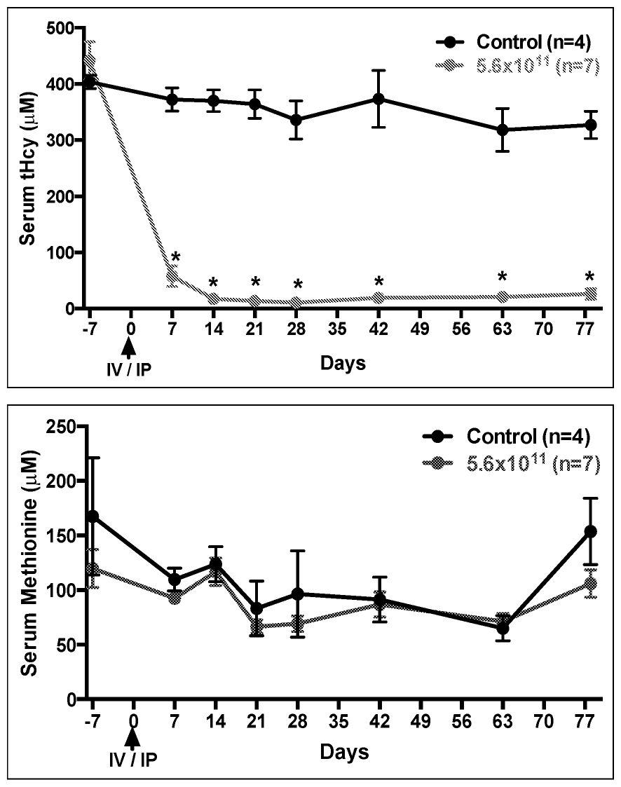

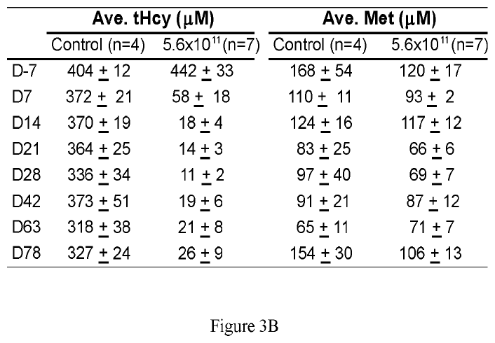

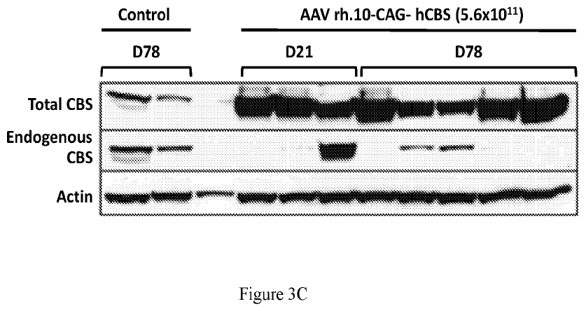

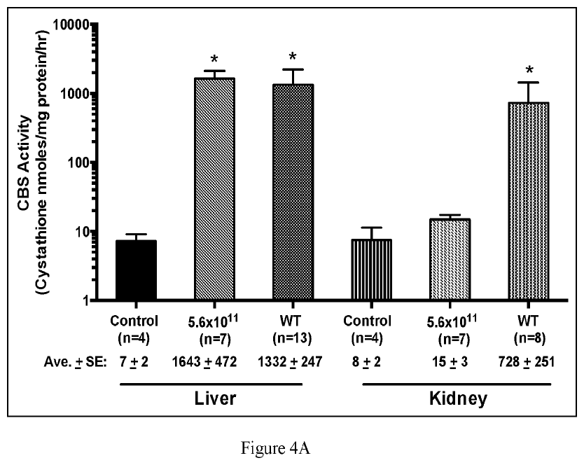

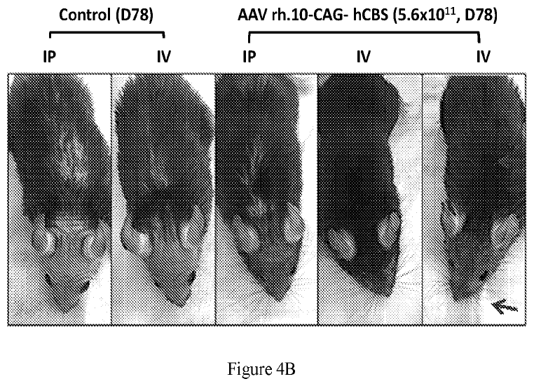

Seven mice injected with the highest dose were followed for up to 77 days (see, FIG. 3A). tHcy showed a >90% decrease from pre-injection levels from D14 through D78. Examination of AAV-encoded CBS protein indicated that protein levels were similar to those observed at D21 (see, FIG. 3B). CBS activity at D78 was similar to that observed in control WT CBS mice (see, FIG. 4A). A noticeably reduced alopecia phenotype was observed in the treated animals (see, FIG. 4B).

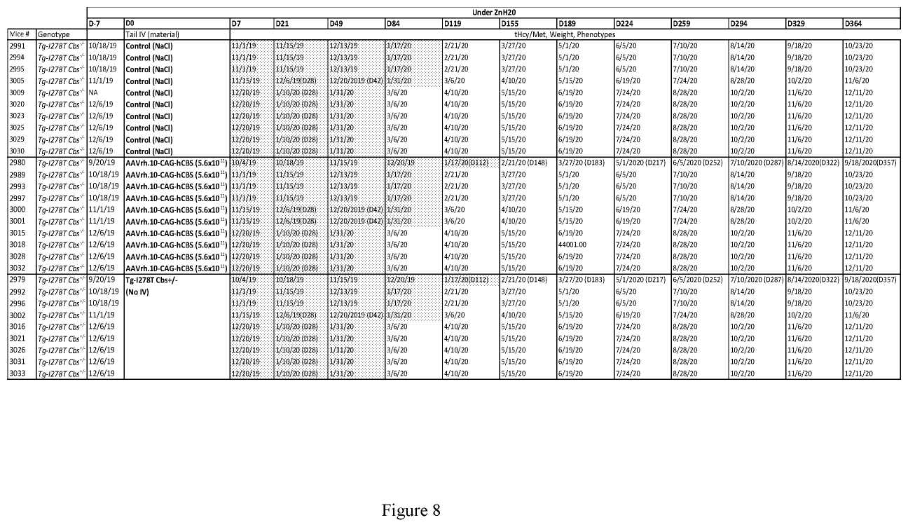

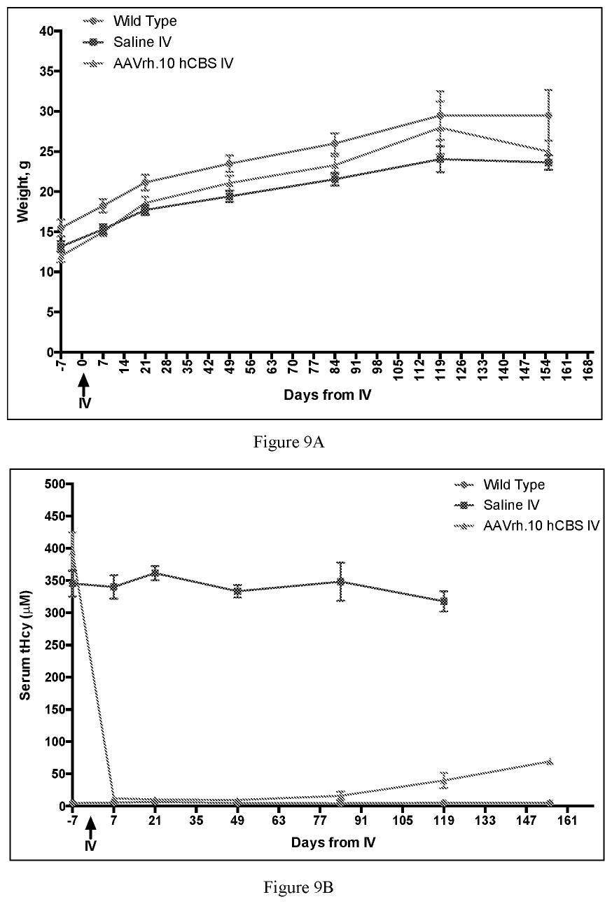

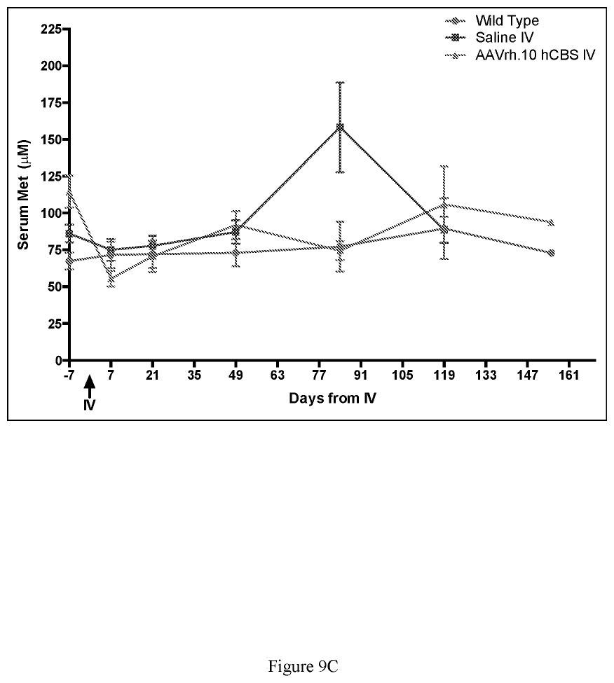

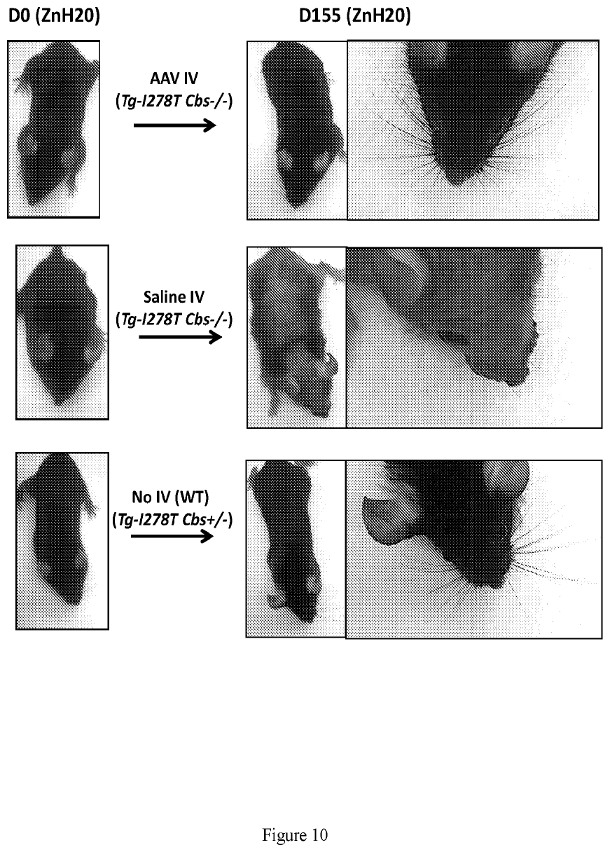

A separate cohort of Tg-I278T Cbs^{-/-} (n=20) and Tg-I278T Cbs^{+/-} (n=9) mice were generated and kept on Zn-water (25 mM ZnSO₄) to induce transgene expression until the termination of the experiment. AAVrh.10-CAG-hCBS (5.6×10″) (n=10) or saline (n=10) was delivered into Tg-I278T Cbs^{-/-} mice via a single lateral vein injection. Three groups of mice, including vector-delivered mice, saline-delivered mice, and Tg-I278T Cbs^{+/-} mice, will be monitored for one year. At the indicated time points (see, FIG. 8), all mice will be monitored for weight changes, serum tHcy, methionine levels, and phenotypic changes in fur and whiskers. After 119 days, AAVrh.10-CAG-hCBS treatment did not affect weight (see, FIG. 9A). Moreover, AAVrh.10-CAG-hCBS treatment resulted in a 94% reduction of serum tHcy levels (20 μM vs. 341 P<0.0001) at D21 that was maintained until at least D49 (see, FIG. 9B) without affecting serum methionine levels (see, FIG. 9C). AAVrh.10-CAG-hCBS also induced correction of phenotypes with thicker whiskers and body hairs, which was maintained for 119 days post-treatment (see, FIG. 10).

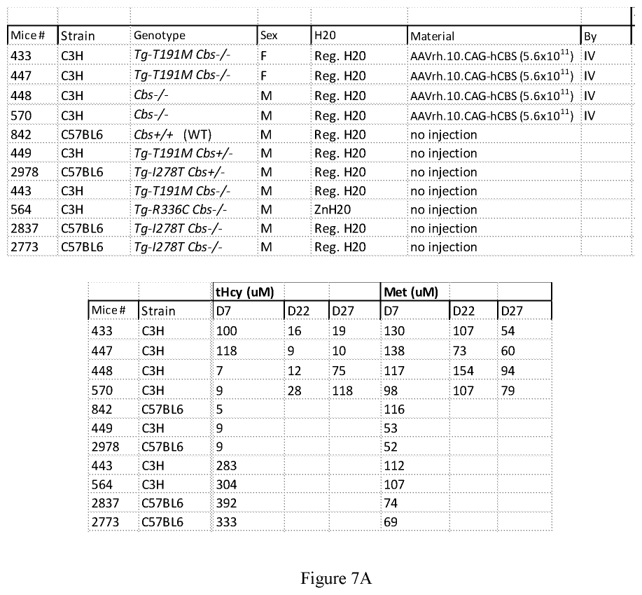

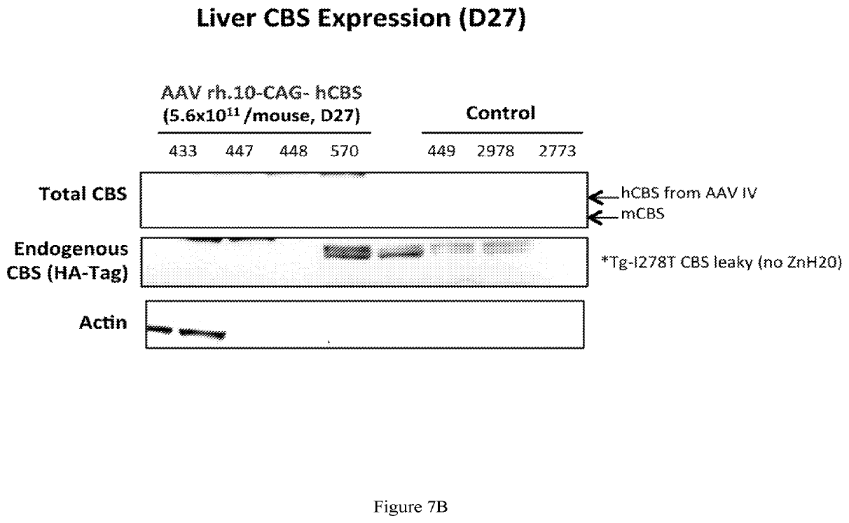

In addition, the above experiments were repeated in T191M CBS^{-/-} C3H strain having a different genetic background from the previously used C57BL6 strain. AAVrh.10-CAG-hCBS (5.6×10¹¹) were delivered into Tg-T191M Cbs^{-/-} (C3H strain background) mice (n=2) and Tg-negative Cbs^{-/-} mice (n=2) via a single lateral vein injection. Serum was prepared from blood collected at D7, D22 and D27 and analyzed for tHcy and methionine (see, FIG. 7A). The sera from wild type mice (Cbs^{+/+} or Cbs^{+/-}) and Cbs^{-/-} in the indicated strain backgrounds was also analyzed. Mice administered vector had a mean tHcy decrease by 83% (Ave. 56 μM vs. 328 μM; P<0.00004) 27 days after injection. Western blot analysis confirmed the expression of vector-derived hCBS protein in the liver tissue (see, FIG. 7B).

In sum, the effectiveness of two different AAV vectors was examined utilizing the Tg-I278T Cbs^{-/-} mouse model of CBS deficiency. Large differences were found in the effectiveness of the different vectors. A virus with the AAVrh.10 serotype containing a CAG promoter driving expression of the hCBS cDNA, however, was highly effective in expressing CBS in the liver and lowering tHcy for an extended period of time. In addition, because the AAVrh.10 serotype is a Rhesus monkey virus, it is expected that it would be less likely to encounter pre-existing neutralizing antibodies in humans.

The data presented herein compares favorably to another approach to treat CBS deficiency, namely enzyme replacement therapy using PEGylated human truncated CBS. Using the exact same Tg-I278T Cbs^{-/-} mouse model described here, Majtan et al. found that they could achieve sustained lowering of tHcy from about 320 μM to about 100 μM with IP injection of 7.5 mg/kg PEG-CBS given 3 times a week (Majtan et al., Molecular Therapy: The Journal of the American Society of Gene Therapy, 2018, 26, 834-844). In the studies described here, however, a greater reduction of tHcy was actually observed. At 77 days after injection, the mean tHcy in the AAVrh.10-CAG-hCBS treated animals was only 26 μM. Also, like the PEG-CBS treated mice, a substantial improvement in the facial alopecia phenotype was observed. Thus, the gene therapy approach described here has the advantage of both greater tHcy lowering and the avoidance of multiple injections. Overall, the results presented herein indicate that AAVrh.10-CAG-hCBS may be a promising approach to treat CBS deficiency.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A recombinant adeno-associated virus (AAV) nucleic acid molecule comprising a CMV early enhancer/chicken beta actin (CAG) promoter operably linked to an exogenous nucleic acid sequence encoding a human Cystathionine β-synthase (hCBS) polypeptide.

2. The recombinant AAV nucleic acid molecule according to claim 1, wherein the exogenous nucleic acid sequence encodes an hCBS polypeptide comprising an amino acid sequence at least 85% identical to the amino acid sequence of SEQ ID NO:1.

3. The recombinant AAV nucleic acid molecule according to claim 1 or claim 2, wherein the exogenous nucleic acid sequence comprises a nucleotide sequence at least 85% identical to the nucleotide sequence of SEQ ID NO:2.

4. The recombinant AAV nucleic acid molecule according to any one of claims 1 to 3, wherein the CAG promoter is upstream of the exogenous nucleic acid sequence encoding the hCBS polypeptide.

5. The recombinant AAV nucleic acid molecule according to any one of claims 1 to 4, wherein the CAG promoter operably linked to the nucleic acid sequence encoding the hCBS polypeptide is surrounded by AAV Inverted Terminal Repeats (ITRs).

6. The recombinant AAV nucleic acid molecule according to any one of claims 1 to 5, wherein the recombinant AAV nucleic acid molecule is present within a plasmid, bacmid, or baculovirus.

7. A method of preparing the recombinant AAV nucleic acid molecule according to any one of claims 1 to 6, comprising:
amplifying the exogenous nucleic acid sequence encoding the hCBS polypeptide from a source containing the exogenous nucleic acid sequence using a pair of primers; and
cloning the amplified exogenous nucleic acid sequence into a pAAV-CAG-containing nucleic acid molecule.

8. The method according to claim 7, wherein the source containing the exogenous nucleic acid sequence is pUC:AHCBS.

9. The method according to claim 7 or claim 8, wherein the pair of primers comprises a first primer comprising the nucleotide sequence 5′-CAGTCTCGAACTTAACATGCCTTCT GAGACCCCC-3′ (SEQ ID NO:3) and a second primer comprising the nucleotide sequence 5′-GGGCCCATTACCGATACTTCACTTCTGGTCCGCTCC-3′ (SEQ ID NO:4).

10. The method according to any one of claims 7 to 9, wherein the pAAV-CAG-containing nucleic acid molecule is pAAV-CAG-MCS.

11. A viral vector encapsidating the recombinant AAV nucleic acid molecule according to any one of claims 1 to 5.

12. The viral vector according to claim 11, wherein the serotype of the AAV vector is AAVrh.10.

13. A method of producing a recombinant AAV vector comprising:
co-transfecting a host cell with CAG-hCB S DNA surrounded by AAV ITRs and a helper nucleic acid molecule that comprises the AAV Rep and Cap sequences and adenovirus helper functions E4, E2a and VA; and
culturing the host cell for a period of time sufficient to produce the recombinant AAV vector.

14. A method of producing a recombinant AAV vector comprising:
co-transfecting a host cell with CAG-hCB S DNA surrounded by AAV ITRs and two helper nucleic acid molecules, the first helper nucleic acid molecule comprising the AAV Rep and Cap sequences, and the second helper nucleic acid molecule comprising the adenovirus helper functions E4, E2a and VA; and
culturing the host cell for a period of time sufficient to produce the recombinant AAV vector.

15. A method of producing a recombinant AAV vector comprising:
transfecting a host cell with CAG-hCB S DNA surrounded by AAV ITRs, wherein the host cell expresses AAV Cap and Rep proteins and adenoviral replication proteins E2, E4, and VA; and
culturing the host cell for a period of time sufficient to produce the recombinant AAV vector.

16. The method according to any one of claims 13 to 15, wherein the host cell is a mammalian cell.

17. The method according to claim 16, wherein the mammalian cell is HEK 293 cell, HEK 293T cell, PerC.6 cell, or any other cell line comprising the Adenovirus E1 helper function.

18. The method according to claim 16 or claim 17, wherein the CAG-hCBS DNA surrounded by AAV ITRs is present within a pAAV-CAG-hCBS plasmid and the helper nucleic acid molecule is a helper plasmid.

19. The method according to claim 18, wherein the helper plasmid is pPAKMArh.10.

20. The method according to any one of claims 13 to 15, wherein the host cell is an insect cell.

21. The method according to 20, wherein the insect cell is Sf9 cell.

22. The method according to claim 20 or claim 21, wherein the CAG-hCBS DNA surrounded by AAV ITRs is present in a baculovirus or a Bacmid.

23. The method according to any one of claims 13 to 22, further comprising obtaining a lysate from the cell.

24. The method according to claim 23, further comprising purifying the viral vector from the lysate.

25. A composition comprising the recombinant AAV nucleic acid molecule according to any one of claims 1-5 and a pharmaceutically acceptable carrier.

26. A composition comprising the viral vector according to claim 11 and a pharmaceutically acceptable carrier.

27. A method of preventing or treating a disease, disorder, or condition associated with elevated homocysteine in a subject in need thereof, comprising administering to the subject the composition according to claim 26.

28. The method according to claim 27, wherein the disease, disorder, or condition associated with elevated homocysteine is CBS deficiency.

29. The method according to claim 27 or claim 28, wherein the composition is administered to the subject by intramuscular injection or intravenous injection.

30. Use of the composition according to claim 26 for the preparation of a medicament for the prevention or treatment of a disease, disorder, or condition associated with elevated homocysteine in a human subject.

31. Use of the composition according to claim 26 for the prevention or treatment of a disease, disorder, or condition associated with elevated homocysteine in a human subject.


SEQUENCE LISTING (5 sequences)

SEQ ID NO: 1 - protein, 551 aa, homo sapien
MPSETPQAEVGPTGCPHRSGPHSAKGSLEKGSPEDKEAKEPLWIRPDAPSRCTWQLGRPASESPHHHTAPAKSPKILPDILKKIGDTPMVRINKIGKKFGLKCELLAKCEFFNAGGSVKDRISLRMIEDAERDGTLKPGDTIIEPTSGNTGIGLALAAAVRGYRCIIVMPEKMSSEKVDVLRALGAEIVRTPTNARFDSPESHVGVAWRLKNEIPNSHILDQYRNASNPLAHYDTTADEILQQCDGKLDMLVASVGTGGTITGIARKLKEKCPGCRIIGVDPEGSILAEPEELNQTEQTTYEVEGIGYDFIPTVLDRTVVDKWFKSNDEEAFTFARMLIAQEGLLCGGSAGSTVAVAVKAAQELQEGQRCVVILPDSVRNYMTKFLSDRWMLQKGFLKEEDLTEKKPWWWHLRVQELGLSAPLTVLPTITCGHTIEILREKGFDQAPVVDEAGVILGMVTLGNMLSSLLAGKVQPSDQVGKVIYKQFKQIRLTDTLGRLSHILEMDHFALVVHEQIQYHSTGKSSQRQMVFGVVTAIDLLNFVAAQERDQK

SEQ ID NO: 2 - DNA, 2583 bp, homo sapien
tcccgggcccgcgacacacgccctcggggtcggtcctcgaggacgcgcagggccccccacccaccaggacgcacgtttcaagctcatcagtaaaggttccttaaattcccgaagggcaagaagttaaccaagtaaaacagcatcggaacaccaggatcccatgacagattctgttgtcacgtctccttacagagtttgagcggtgctgaactgtcagcaccatctgtccggtcccagcatgccttctgagaccccccaggcagaagtggggcccacaggctgcccccaccgctcagggccacactcggcgaaggggagcctggagaaggggtccccagaggataaggaagccaaggagcccctgtggatccggcccgatgctccgagcaggtgcacctggcagctgggccggcctgcctccgagtccccacatcaccacactgccccggcaaaatctccaaaaatcttgccagatattctgaagaaaatcggggacacccctatggtcagaatcaacaagattgggaagaagttcggcctgaagtgtgagctcttggccaagtgtgagttcttcaacgcgggcgggagcgtgaaggaccgcatcagcctgcggatgattgaggatgctgagcgcgacgggacgctgaagcccggggacacgattatcgagccgacatccgggaacaccgggatcgggctggccctggctgcggcagtgaggggctatcgctgcatcatcgtgatgccagagaagatgagctccgagaaggtggacgtgctgcgggcactgggggctgagattgtgaggacgcccaccaatgccaggttcgactccccggagtcacacgtgggggtggcctggcggctgaagaacgaaatccccaattctcacatcctagaccagtaccgcaacgccagcaaccccctggctcactacgacaccaccgctgatgagatcctgcagcagtgtgatgggaagctggacatgctggtggcttcagtgggcacgggcggcaccatcacgggcattgccaggaagctgaaggagaagtgtcctggatgcaggatcattggggtggatcccgaagggtccatcctcgcagagccggaggagctgaaccagacggagcagacaacctacgaggtggaagggatcggctacgacttcatccccacggtgctggacaggacggtggtggacaagtggttcaagagcaacgatgaggaggcgttcacctttgcccgcatgctgatcgcgcaagaggggctgctgtgcggtggcagtgctggcagcacggtggcggtggccgtgaaggccgcgcaggagctgcaggagggccagcgctgcgtggtcattctgcccgactcagtgcggaactacatgaccaagttcctgagcgacaggtggatgctgcagaagggctttctgaaggaggaggacctcacggagaagaagccctggtggtggcacctccgtgttcaggagctgggcctgtcagccccgctgaccgtgctcccgaccatcacctgtgggcacaccatcgagatcctccgggagaagggcttcgaccaggcgcccgtggtggatgaggcgggggtaatcctgggaatggtgacgcttgggaacatgctctcgtccctgcttgccgggaaggtgcagccgtcagaccaagttggcaaagtcatctacaagcagttcaaacagatccgcctcacggacacgctgggcaggctctcgcacatcctggagatggaccacttcgccctggtggtgcacgagcagatccagtaccacagcaccgggaagtccagtcagcggcagatggtgttcggggtggtcaccgccattgacttgctgaacttcgtggccgcccaggagcgggaccagaagtgaagtccggagcgctgggcggtgcggagcgggcccgccacccttgcccacttctccttcgctttcctgagccctaaacacacgcgtgattggtaactgcctggcctggcaccgttatccctgcacacggcacagagcatccgtctcccctcgttaacacatggcttcctaaatggccctgtttacggcctatgagatgaaatatgtgattttctctaatgtaacttcctcttaggatgtttcaccaaggaaatattgagagagaagtcggccaggtaggatgaacacaggcaatgactgcgcagagtggattaaaggcaaaagagagaagagtccaggaaggggcggggagaagcctgggtggctcagcatcctccacgggctgcgccgtctgctcggggctgagctggcgggagcagtttgcgtgtttgggttttttaattgagatgaaattcaaataacctaaaaatcaatcacttgaaagtgaacaatcagcggcatttagtacatccagaaagttgtgtaggcaccacctctgtcacgttctggaacattctgtcatcaccccgtgaagcaatcatttcccctcccgtcttcctcctcccctggcaactgctgatcgactttgtgtctctgttgtctaaaataggttttccctgttctggacatttcatataaatggaatcacacaa

SEQ ID NO: 3 - DNA, 34 bp, artificial sequence
  primer
cagtctcgaacttaacatgccttctgagaccccc

SEQ ID NO: 4 - DNA, 36 bp, artificial sequence
  primer
gggcccattaccgatacttcacttctggtccgctcc

SEQ ID NO: 5 - DNA, 5320 bp, artificial sequence
  pAAV-CAG-MCS construct
  misc_feature (1968)..(1970): n is a, c, g, t or u
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccgctctagaactagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgagctcggtacccggggatcctctagagtcgacctgcaggaattcgagctcctaggatatcaattgttaattaagnnnaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttatcgataccgtcgacctcgagggggggcccggtacccagcttttgttccctttgcggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgggccgcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggcggggatcctctagagtcgacctgcaggcatgcaagcttcagcggcccatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggcctttttacggttcctggccttttgctggccttttgctcacatgtc